(19) United States
(12) Patent Application Publication
(10) Pub. No.: US 2026/0218306 A1
(43) Pub. Date: Jul. 30, 2026

(54) METHODS FOR DETECTING GLIOBLASTOMA IN EXTRACELLULAR VESICLES

(71) Applicant: Regents of the University of Minnesota, Minneapolis, MN (US)

(72) Inventor: Okay SAYDAM, Minneapolis, MN (US)

(73) Assignee: Regents of the University of Minnesota, Minneapolis, MN (US)

(21) Appl. No.: 19/141,948

(22) PCT Filed: Dec. 19, 2023
(86) PCT No.: PCT/US2023/084880

§ 371 Date: Jun. 20, 2025

Related U.S. Application Data
(60) Provisional application No. 63/434,904, filed on Dec. 22, 2022.

(51) Int. Cl. C12Q 1/6886; G01N 15/1429

(52) U.S. Cl. CPC C12Q 1/6886; G01N 15/1429; C12Q 2600/154; C12Q 2600/156; C12Q 2600/158

(57) ABSTRACT

In certain embodiments, the present invention provides a panel of probes associated with glioblastoma that hybridize to nucleic acid and kits that comprise a collection of panels of probes associated with glioblastoma. In certain embodiments, the present invention provides a method of detecting the presence of biomarkers associated with an increased risk of glioblastoma in a human subject. In certain embodiments, the present invention provides a method of treating a human subject for glioblastoma.

### RELATED APPLICATIONS

This application claims priority to U.S. Provisional Application No. 63/434,904 that was filed on Dec. 22, 2022. The entire content of the application referenced above is hereby incorporated by reference herein.

### BACKGROUND

The field of the invention is related to cancer diagnostics and treatment. More particularly, the invention relates to exosomes and/or other extracellular vesicles (EVs) specific for cancer.

Molecular profiling of tumors obtained from individual patients improves the selection of personalized cancer treatment therapies, patient responses, detection of drug resistance, and monitoring of tumor relapse. Profiling tumors generally involves obtaining resected tumor samples by invasive surgeries. The limitations to such invasive procedures include difficulty in acquiring tumor samples for both tumor quantity and quality. Another drawback is that acquiring biopsy samples by invasive methods throughout treatment to monitor tumor response and relapse pose major challenges in tumor profiling. A further limitation to invasive sampling methods is the heterogeneity of resected tumor samples as a whole. Further, in the case of metastasis, where tumors have spread and constantly evolve both spatially and temporally in response to treatment over time, multiple biopsies may be required. These challenges make it difficult to obtain a holistic image of a tumor.

Recently, new non-invasive techniques are being developed to address these limitations, such as liquid biopsy (LB). Liquid biopsies consist of isolating tumor-derived entities like circulating tumor cells, circulating tumor DNA, tumor extracellular vesicles, etc., present in the body fluids of patients with cancer, followed by an analysis of genomic and proteomic data contained within them. Liquid biopsies methods permit continuous monitoring by repeated sampling. Further, LB provides enhanced sensitivity in diagnosis and ease of repeated sampling throughout treatment much more conveniently and non-invasively.

### SUMMARY

One aspect provides a panel of probes that hybridize to nucleic acid, the panel comprising at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1).

One aspect provides a panel of probes that hybridize to nucleic acid, the panel comprising at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8 and AC008964.1 (Table 2).

One aspect provides a panel of probes that hybridize to nucleic acid, the panel comprising at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4).

One aspect provides a panel of probes that hybridize to nucleic acid, the panel comprising at least 2 probes specific for TERT mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1 (Table 3).

One aspect provides a kit comprising a collection of probes, wherein the collection comprises a panel of probes, and instructions for use in analyzing a biological sample. In certain embodiments, the panel comprises at least 2-3 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1). In certain embodiments, the panel comprises at least 2-3 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8, and AC008964.1 (Table 2). In certain embodiments, the panel comprises at least 2-3 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4). In certain embodiments, the panel comprises at least 2-3 probes specific for TERT mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1 (Table 3).

One aspect provides a method of detecting the presence of biomarkers associated with an increased risk of glioblastoma in a human subject, comprising:
(i) contacting DNA from a biological sample containing cells from the subject with the panel of probes to form hybridized target sequences;
(ii) detecting the hybridized target sequences;
(iii) determining the number of hybridized target sequences detected; and
(iv) indicating that the human subject has an increased risk of glioblastoma if more than 2 biomarkers are present. In certain embodiments, the panel comprises at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1). In certain embodiments, the panel comprises at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8, and AC008964.1 (Table 2). In certain embodiments, the panel comprises at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4). In certain embodiments, the panel comprises at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2 and LARGE-IT1 (Table 3).

One aspect provides a method of treating a human subject for glioblastoma, comprising:
(i) contacting DNA from a biological sample containing cells from the subject with the panel of probes of any one of claims 1-7 to form hybridized target sequences;
(ii) detecting the hybridized target sequences;
(iii) determining the number of hybridized target sequences detected;
(iv) indicating that the human subject has an increased risk of glioblastoma if more than 2 biomarkers are present; and
(v) administering an appropriate treatment to the patient. In certain embodiments, the panel comprises at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1). In certain embodiments, the panel comprises at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8, and AC008964.1 (Table 2). In certain embodiments, the panel comprises at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4). In certain embodiments, the panel comprises at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1 (Table 3).

One aspect provides a method of detecting glioblastoma in a patient comprising:
(a) obtaining a sample from the patient;
(b) isolating extracellular vesicles from the sample; and
(c) detecting expression of at least one cancer marker in the isolated extracellular vesicles.

### BRIEF DESCRIPTION OF DRAWINGS

FIGS. 1A-1B. FIG. 1A provides a heat map of the Differentially expressed genes (DEGs) with an absolute fold-change of 2 and an FDR-adjusted p value of less than 0.05. FIG. 1B provides a list of the DEG.

FIGS. 2A-2D. (FIG. 2A) Venn diagram of all dysregulated genes found in glioblastoma subgroups compared to controls. (FIG. 2B) Table of genes dysregulated in one glioblastoma subgroup compared to controls. (FIG. 2C) Table of genes dysregulated in two glioblastoma subgroups compared to controls. (FIG. 2D) Table of genes dysregulated in three or more glioblastoma subgroups compared to controls.

FIGS. 3A-3B. Glioblastoma IDH-wt cancer panel. ROC and Precision recall curves for IDH1 WT GBM vs controls genes. FIG. 3A) Receiver operating characteristics (ROC) curve and FIG. 3B) Precision-recall curve are shown for IDH1 wild-type vs control samples. Lasso-penalized binomial regression model was used employing 24 of 569 differentially expressed genes as predictors, resulting in 96% sensitivity and 80% specificity in detection of IDH1 wild-type glioblastomas in comparison to control samples. Training data (dark line) and test data (lighter “stair-step” line) are shown. Dotted black lines represent performance of models generated from 100 random permutations of the response variable.

FIGS. 4A-4B. Glioblastoma TERT promoter mutation cancer panel. ROC and Precision recall curves for TERT promoter mutation vs control genes. FIG. 4A) Receiver operating characteristics (ROC) curve and FIG. 4B) Precision-recall curve are shown for the prediction of TERT promoter mutation status. Lasso-penalized binomial regression model was used employing 20 of 569 differentially expressed genes as predictors, which resulted in 89% sensitivity and 100% specificity in predicting TERT promoter mutation. Training data (dark line) and test data (lighter line) are shown. Dotted black lines represent performance of models generated from 100 random permutations of the response variable.

FIGS. 5A-5B. ROC and Precision recall curves for MGMT promoter methylated vs control genes. Glioblastoma MGMT promoter methylated cancer panel. FIG. 5A) Receiver operating characteristics (ROC) curve and FIG. 5B) Precision-recall curve are shown for the prediction of MGMT promoter methylation status. Lasso-penalized binomial regression model was used employing 17 of 569 differentially expressed genes as predictors, which resulted in 91% sensitivity and 73% specificity in predicting MGMT methylation status. Training data (dark line) and test data (lighter line) are shown. Dotted black lines represent performance of models generated from 100 random permutations of the response variable.

FIGS. 6A-6B. Glioblastoma TP53 mutation cancer panel. ROC and Precision recall curves for TP53 mutation vs control genes. FIG. 6A) Receiver operating characteristics (ROC) curve and FIG. 6B) Precision-recall curve are shown for the prediction of TP53 mutation status. Lasso-penalized binomial regression model was used employing 15 of 569 differentially expressed genes as predictors, which resulted in 100% sensitivity and 89% specificity in predicting TP53 mutation status. Training data (dark line) and test data (lighter line) are shown. Dotted black lines represent performance of models generated from 100 random permutations of the response variable.

### DETAILED DESCRIPTION

Glioblastoma is one of the most devastating neoplasms of the central nervous system. The present inventors focused on the development of serum extracellular vesicle (EV)-based glioblastoma tumor marker panels that can be used in the clinic to diagnose glioblastomas and to monitor tumor burden, progression, and regression in response to treatment. RNA sequencing studies were performed using RNA isolated from serum EVs of both patient (n=) and control donors (n=31). RNA sequencing results for preoperative glioblastoma EVs compared to control EVs revealed 569 differentially expressed genes (DEGs, 2XFC, FDR<0.05). Using these DEGs, serum EV-based biomarker panels were developed for the following: wild-type IDH1 status (96% sensitivity/80% specificity), MGMT promoter methylation (91% sensitivity/73% specificity), mutation in the p53 gene (100% sensitivity/89% specificity), and TERT promoter mutation (89% sensitivity/100% specificity). This is the first study showing that serum EV-based biomarker panels can diagnose glioblastomas with high sensitivity and specificity.

Embodiments of the present disclosure provide the use of extracellular vesicles and exosomes for cancer detection and diagnosis, cancer targeting and treatment. In one embodiment, the present disclosure describes a newly identified list of proteins differentially contained in cancer cell EVs/exosomes versus the normal cell EVs/exosomes The inventors have also developed a novel method of detecting and profiling circulating exosomes and EVs from a subject at single vesicle levels in order to detect, diagnose, and monitor disease development and progression. In one example, an expression profile of proteins on EVs and exosomes from patients with cancer can be used to distinguish the population with cancer from the healthy controls.

EVs are cell-derived vesicles with a closed double-layer membrane structure. According to their size and density, EVs mainly include exosomes (30-150 nm), micro vesicles (MVs) (100-1000 nm), and apoptotic bodies or cancer related oncosomes (1-10 μm). Exosomes include multi-vesicle body (MVB)-derived EVs carrying specific markers. EVs exist in virtually all body fluids of human, animals, bacteria, and plants, such as blood, urine, saliva, beer, milk, etc.

EVs and exosomes are able to carry various molecules, such as proteins, lipids and RNAs on their surface as well as within their lumen. The EV and exosomal surface proteins can mediate organ-specific homing of circulating EVs and exosomes. As used herein, the term “extracellular vesicles” or “EVs” includes all cell-derived vesicles with a closed double-layer membrane structure derived from multivesicular bodies or from the plasma membrane, including exosomes, microvesicles, and oncocomes.

The contents of EVs and exosomes are able to serve as novel biomarkers for assisting in the diagnosis, prognosis, and prediction of human diseases, such as cancer. The methods described herein may be used to monitor dynamic changes of exosome and EV contents to provide new ways of monitoring diseases.

Approaches to detect and characterize exosomes and other EVs include: (1) electron microscopy (EM) to assess structure and size; (2) nanoparticle tracking analysis (NTA) to reveal size and zeta potential; (3) protein analysis via immunofluorescence staining, western blotting, ELISA, and mass spectrometry; (4) RNA analysis using array platforms, RNA sequencing, and PCR; and (5) analysis of lipids, sugar, and other components by biochemical assays. Among these approaches, EM provides high-resolution imaging but is neither convenient nor affordable for high throughput molecular profiling of large numbers of circulating exosorne samples for potential clinical applications. NTA utilizes light scattering and Brownian motionto measure particle size but does not differentiate between vesicles within a size range of 5 orders of magnitude due to the low dynamic range of the camera. In addition, NTA is not suitable for molecular profiling of exosomes because of low sensitivity to fluorescent signals. While all the EV or exosomal components potentially serve as molecular biomarkers of circulating vesicles in human disease, it is pivotal and necessary to improve high throughput profiling of surface molecules such as proteins in exosomes and other EVs, which could be readily detectable and serve as clinically relevant biomarkers. The present invention provides a method for readily detecting and profiling surface molecules of EVs and exosomes which can be used as biomarkers for specific diseases, specifically cancer.

The present invention provides a rapid and high throughput profiling of surface molecules at a single exosome/EV level. Although flow cytometry is a commonly used optical method to analyze cells based on the light scattering and fluorescence-activated mechanisms, conventional flow cytometers are only capable of detecting particles at a minimal size of 200-500 nm that is beyond the size of exosomes and small MTVs. In addition, they are ineffective at discriminating particles that differ by 100-200 nm or less. In conventional flow cytometry, the background signal is often high in the <200 nm size range, due to contaminating particles in the sheath buffer. Furthermore, the detectable level of immunolabeling signal is limiting in such small particles. Recently, latex beads in micrometer sizes have been used to bind to multiple exosomes to enhance the ability to detect exosomes stained with fluorophore-conjugated antibodies by conventional flow cytometry. However, this bead-based approach does not provide single exosome profiling and therefore fails to discriminate between different subsets of exosomes, which may result in the loss of distinctive signatures with potential diagnostic importance. The present invention provides methods of detecting and profiling circulating exosomes and EVs from a subject at single vesicle levels in order to detect, diagnose and monitor disease development and progression. In one example, an expression profile of proteins on EVs and exosomes from patients with cancer can be used to distinguish the population with cancer from the healthy controls.

In one embodiment, the disclosure provides a method of detecting cancer specific exosomes/EVs from patients comprising: (a) obtaining a sample from the patient; (b) isolating the extracellular vesicles from the sample; and (c) detecting expression of at least one cancer marker in the isolated EVs. In one embodiment, the extracellular vesicles are exosomes.

The terms “subject” and “patient” are used interchangeably and refer to any animal (e.g., a mammal), including, but not limited to, humans, non-human primates, rodents, and the like, which is to be the recipient of a particular treatment. Typically, the terms “subject” and “patient” are used interchangeably herein in reference to a human subject. In a preferred embodiment, the subject is a human having or suspected of having cancer.

The term sample refers to a sample obtained from a subject. Suitable samples include a body fluid sample, such as, for example, blood, urine, cerebral spinal fluid, plasma, breast milk, saliva, or tissue samples (biopsy sample, tumor sample, breast tumor, other tumor tissues or normal tissues, among others).

In some embodiments, the EVs or exosomes that are isolated express one or more proteins that are expressed on cancer cells. Suitable markers for glioblastoma include the markers listed in Tables 1-4 below:

TABLE 1
IDH1WT_vs_control_LASSO_genes

| var | val | gene_name | gene_biotype | chromosome |
|---|---|---|---|---|
| (Intercept) | 1.96124128 | | | |
| ENSG00000259366 | 0.44042476 | AC108449.2 | lncRNA | 8 |
| ENSG00000199994 | −0.3821842 | RNA5SP145 | rRNA_pseudogene | 3 |
| ENSG00000286155 | 0.36808662 | FP671120.11 | TEC | 21 |
| ENSG00000273258 | −0.3643707 | AC016737.2 | lncRNA | 2 |
| ENSG00000283312 | 0.35825476 | AC017104.4 | processed_pseudogene | 2 |
| ENSG00000231660 | −0.3105246 | RPS8P6 | processed_pseudogene | 3 |
| ENSG00000286817 | 0.28536854 | AC105339.6 | lncRNA | 15 |
| ENSG00000234779 | −0.2151196 | BNC2-AS1 | lncRNA | 9 |
| ENSG00000228384 | −0.2143883 | AC007040.1 | lncRNA | 2 |
| ENSG00000278877 | 0.21085591 | AC091932.2 | TEC | 5 |
| ENSG00000226716 | 0.1977556 | AL589986.1 | lncRNA | 1 |
| ENSG00000236348 | 0.15795008 | PSMC1P10 | processed_pseudogene | 2 |
| ENSG00000287386 | 0.14936478 | AC009248.3 | lncRNA | 12 |
| ENSG00000240350 | 0.12400534 | SOCAR | lncRNA | 2 |
| ENSG00000163191 | 0.1160908 | S100A11 | protein_coding | 1 |
| ENSG00000100564 | −0.0767305 | PIGH | protein_coding | 14 |
| ENSG00000256357 | 0.0486148 | AL132708.1 | lncRNA | 14 |
| ENSG00000196976 | −0.044436 | LAGE3 | protein_coding | X |
| ENSG00000274104 | 0.04374432 | AC020910.5 | lncRNA | 19 |
| ENSG00000196860 | −0.0354077 | TOMM20L | protein_coding | 14 |
| ENSG00000258670 | −0.0242997 | AL049874.3 | lncRNA | 14 |
| ENSG00000251624 | −0.0153749 | UNC93B7 | unprocessed_pseudogene | 4 |
| ENSG00000221869 | 0.01083785 | CEBPD | protein_coding | 8 |
| ENSG00000169075 | 0.00408832 | Z99496.1 | processed_pseudogene | 6 |

TABLE 2
TP53mut vs control LASSO genes

| var | val | gene_name | gene_biotype | chromosome |
|---|---|---|---|---|
| (Intercept) | 3.41175471 | | | |
| ENSG00000199523 | −0.8947731 | RNA5SP226 | rRNA_pseudogene | 6 |
| ENSG00000224014 | 0.46170202 | AL390728.3 | processed_pseudogene | 1 |
| ENSG00000259944 | 0.31072702 | CDRT7 | lncRNA | 17 |
| ENSG00000199994 | −0.310697 | RNA5SP145 | rRNA_pseudogene | 3 |
| ENSG00000259366 | 0.28147501 | AC108449.2 | lncRNA | 8 |
| ENSG00000228209 | 0.25804971 | PNPP1 | processed_pseudogene | 2 |
| ENSG00000197364 | 0.22641151 | S100A7L2 | protein_coding | 1 |
| ENSG00000196704 | −0.1733978 | AMZ2 | protein_coding | 17 |
| ENSG00000268509 | −0.1394895 | AC026202.3 | lncRNA | 3 |
| ENSG00000233167 | 0.13887579 | EEF1A1P26 | processed_pseudogene | 7 |
| ENSG00000232081 | 0.05797308 | LARGE-IT1 | lncRNA | 22 |
| ENSG00000248396 | 0.02861405 | TOMM22P4 | processed_pseudogene | 4 |
| ENSG00000128045 | −0.022203 | RASL11B | protein_coding | 4 |
| ENSG00000278196 | 0.01956298 | IGLV2-8 | IG_V_gene | 22 |
| ENSG00000286809 | 0.00442883 | AC008964.1 | lncRNA | 5 |

TABLE 3
TERTmut vs. control LASSO

| var | val | gene_name | gene_biotype | chromosome |
|---|---|---|---|---|
| (Intercept) | 3.844234 | | | |
| ENSG00000234779 | −0.4758844 | BNC2-AS1 | lncRNA | 9 |
| ENSG00000283312 | 0.46945706 | AC017104.4 | processed_pseudogene | 2 |
| ENSG00000287386 | 0.46816802 | AC009248.3 | lncRNA | 12 |
| ENSG00000259366 | 0.45604176 | AC108449.2 | lncRNA | 8 |
| ENSG00000228384 | −0.4314381 | AC007040.1 | lncRNA | 2 |
| ENSG00000199994 | −0.3767563 | RNA5SP145 | rRNA_pseudogene | 3 |
| ENSG00000196976 | −0.3362525 | LAGE3 | protein_coding | X |
| ENSG00000253697 | −0.2691059 | AC103834.1 | unprocessed_pseudogene | 8 |
| ENSG00000251624 | −0.2461036 | UNC93B7 | unprocessed_pseudogene | 4 |
| ENSG00000273258 | −0.22221 | AC016737.2 | lncRNA | 2 |
| ENSG00000228209 | 0.19318085 | PNPP1 | processed_pseudogene | 2 |
| ENSG00000262107 | 0.16466365 | MTND6P33 | unprocessed_pseudogene | 16 |
| ENSG00000286155 | 0.13590366 | FP671120.11 | TEC | 21 |
| ENSG00000224363 | 0.12368316 | FP700111.1 | lncRNA | 1 |
| ENSG00000196860 | −0.1043729 | TOMM20L | protein_coding | 14 |
| ENSG00000271966 | −0.0657957 | AC021321.1 | lncRNA | 8 |
| ENSG00000240350 | 0.05405423 | SOCAR | lncRNA | 2 |
| ENSG00000243352 | −0.0454639 | RN7SL8P | misc_RNA | 7 |
| ENSG00000207005 | 0.04384231 | RNU1-2 | snRNA | 1 |
| ENSG00000232081 | 0.0235495 | LARGE-IT1 | lncRNA | 22 |

TABLE 4
MGMTmeth vs. control LASSO genes

| var | val | gene_name | gene_biotype | chromosome |
|---|---|---|---|---|
| (Intercept) | 0.88353743 | | | |
| ENSG00000199523 | −0.2902019 | RNA5SP226 | rRNA_pseudogene | 6 |
| ENSG00000278196 | 0.25510607 | IGLV2-8 | IG_V_gene | 22 |
| ENSG00000262107 | 0.23426246 | MTND6P33 | unprocessed_pseudogene | 16 |
| ENSG00000259366 | 0.20827857 | AC108449.2 | lncRNA | 8 |
| ENSG00000234965 | −0.2061712 | SHISA8 | protein_coding | 22 |
| ENSG00000267472 | −0.1949286 | ARHGAP27P2 | processed_pseudogene | 17 |
| ENSG00000224014 | 0.17641215 | AL390728.3 | processed_pseudogene | 1 |
| ENSG00000236348 | 0.10951381 | PSMC1P10 | processed_pseudogene | 2 |
| ENSG00000283312 | 0.07222801 | AC017104.4 | processed_pseudogene | 2 |
| ENSG00000251624 | −0.0635941 | UNC93B7 | unprocessed_pseudogene | 4 |
| ENSG00000281849 | −0.0509602 | AL732314.8 | lncRNA | X |
| ENSG00000227220 | −0.0441041 | AL133346.1 | lncRNA | 6 |
| ENSG00000228209 | 0.03837699 | PNPP1 | processed_pseudogene | 2 |
| ENSG00000256357 | 0.02640682 | AL132708.1 | lncRNA | 14 |
| ENSG00000207005 | 0.02371938 | RNU1-2 | snRNA | 1 |
| ENSG00000199994 | −0.0065077 | RNA5SP145 | rRNA_pseudogene | 3 |
| ENSG00000272858 | 0.0030647 | Z93930.3 | lncRNA | 22 |

Suitably, in one method, the proteins that are preferentially expressed in cancer EVs are determined or identified by comparing EVs isolated from patients having cancer with EVs isolated from healthy, non-cancerous patients. In some embodiments, comparing EVs of healthy and cancer patients allows for identification of an EV protein profile that is associated with such cancer.

Suitable methods of isolating EVs and exosomes are known in the art and include, but are not limited to, for example ultracentrifugation or exosome isolation kits which are commercially available (e.g. Total Exosome Isolation Kit from ThermoFisher Scientific).

In one embodiment, the EVs are isolated from a sample using beads or microspheres. The beads or microspheres are allowed to bind to the EVs and an antibody specific to a cancer antigen can be used for flow analysis of the sample. In some embodiments, head-assisted flow cytometry is used to characterize the EVs derived from the subject.

EVs may be identified by the expression of one or more exosomal markers on the exosomes surface. Suitable exosomal markers include, but are not limited to, for example, CD63, CD81, CD9, LAMP2B, Tsg101, or Alix. In some embodiments, the methods are used with extracellular vesicles besides exosomes, in which case one or more of the exosome-specific markers may not be present.

In some embodiments, the detection of one or more cancer cell marker on the one or more EVs is by the novel micro flow cytometer (MFC) described in the Example which is performed in an automatic, sensitive, and high throughput manner, wherein the protein expression on individual EVs/exosomes is quantitatively measured and its association with cancer status analyzed. The MFC complements systemic mass spectrometry analysis, RNA sequencing, low-throughput but high resolution TEM known in the art.

In some embodiments, step (c) comprises determining a differential expression profile for at least one cancer marker in the samples from patients having cancer as compared to control healthy population known to not have cancer. In some embodiments, the differential expression profile includes at least two cancer cell markers, alternatively at least three cancer cell markers.

The methods described herein can be used for the detection, diagnosis, targeting, and treatment of a subject having cancer, in particular, glioblastoma.

In certain embodiments, patients have or are suspected of having cancer. The circulating exosome/EV profiling approaches at single vesicle levels and collective levels (mass spectrometry) as described herein can be used for the identification of surface markers associated with diagnoses, prognoses, and treatment of cancer. In one embodiment, the cancer is glioblastoma.

In some embodiments, the cancer stem cell markers on individual EVs/exosomes within the isolated EVs/exosomes are detected. Suitable methods of detecting markers on individual exosomes include but are not limited to micro flow cytometry as described herein. This novel method of micro flow cytometry allows for the detection of markers on individual exosomes and EVs. This new method has advantages over prior methods of detecting EVs, such as florescent microscopy (FM), Transmission Electron Microscopy (TEM), nanoparticle tracking analysis (NTA). While these methods may also be used to detect EVs, these methods are time consuming, expensive for high-throughput molecular profiling of large number of circulating exosomes. FM is time consuming and provides false positive signal. TEM provides high-resolution imaging but is neither convenient nor affordable for high-throughput molecular profiling of large numbers of circulating exosome samples for potential clinical applications. NTA requires a very specific density of nanoparticles and it is not suitable for molecular profiling of exosomes. It is only with the methods of the present invention that a fast, high throughput system of profiling individual EVs has been developed.

### Definitions

Unless otherwise indicated, the practice of the method and system disclosed herein involves conventional techniques and apparatus commonly used in molecular biology, microbiology, protein purification, protein engineering, protein and DNA sequencing, and recombinant DNA fields, which are within the skill of the art. Such techniques and apparatus are known to those of skill in the art and are described in numerous texts and reference works (See e.g., Sambrook et al., “Molecular Cloning: A Laboratory Manual,” Third Edition (Cold Spring Harbor), [2001]); and Ausubel et al., “Current Protocols in Molecular Biology” [1987]).

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein.

Every numerical range given throughout this specification will include every narrower numerical range that falls within such broader numerical range, as if such narrower numerical ranges were all expressly written herein.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art. As used herein, the singular terms “a,” “an,” and “the” include the plural reference unless the context clearly indicates otherwise.

Unless otherwise indicated, nucleic acids are written left to right in 5′ to 3′ orientation and amino acid sequences are written left to right in amino to carboxy orientation, respectively.

A sequence of interest as used herein indicates a nucleic acid sequence in a genome of an organism such as a human. In some implementations, the sequence of interest is a gene, a SNP, an exon, a regulatory sequence of a gene, etc. In some implementations, the sequence of interest is a chromosome or a sub-chromosomal region.

A variant of interest is particular variant of a genetic sequence that is to be measured, qualified, quantified, or detected. In some implementations, a variant of interest is a variant known or suspected to be associated with a condition, such as a cancer, a tumor, or a genetic disorder.

A gene is a locus (or region) of DNA which is made up of nucleotides and is the molecular unit of heredity.

Genes can acquire mutations in their sequence, leading to different variants, known as alleles, in the population. These alleles encode slightly different versions of a protein, which cause different phenotype traits.

Allele frequency or gene frequency, is the frequency of an allele of a gene (or a variant of the gene) relative to other alleles of the gene, which can be expressed as a fraction or percentage. An allele frequency is often associated with a particular genomic locus, because a gene is often located at with one or more locus. However, an allele frequency as used herein can also be associated with a size-based bin of DNA fragments. In this sense, DNA fragments containing an allele are assigned to different size-based bins. The frequency of the allele in a size-based bin relative to the frequency of other alleles is an allele frequency. In some implementations, the frequency of an allele or a variant is a proportion of reads supporting the variant calls out of all reads in multiple bins, such as a prioritized set of bins.

The term “parameter” herein refers to a numerical value that characterizes a property of a system such as a physical feature whose value or other characteristic has an impact on a relevant condition such as a sample or DNA fragments having a simple nucleotide variant or a copy number variant. In some cases, the term parameter is used with reference to a variable that affects the output of a mathematical relation or model, which variable may be an independent variable (i.e., an input to the model) or an intermediate variable based on one or more independent variables. Depending on the scope of a model, an output of one model may become an input of another model, thereby becoming a parameter to the other model.

The term “fragment size parameter” refers to a parameter that relates to the size or length of a fragment or a collection of fragments such nucleic acid fragments; e.g., a fragment obtained from a bodily fluid. A fragment size or size range may be a characteristic of an aberrant genome or a portion thereof when the genome produces nucleic acid fragments having a higher concentration of the size or size range relative to nucleic acid fragments from another genome or another portion of the same genome. Various implementations disclosed herein provide methods to combine size information with sequence information to determine simple nucleotide variants. Additionally, the abundance of sequences can also be combined with size information to determine a structural variation or a copy number variation. Various implementations combine fragment size information and sequence information in innovative ways that are more efficient than simple additions or alternative selections of the two kinds of information, thereby providing improved performance over conventional assays for detecting cancer variants having low variation.

The term “potentially variant-containing fragment” is used herein to refer to fragments that are identified as fragments that are suspected of harboring a sequence mutation corresponding to a cancer variant. In various implementations, a fragment is identified as a potentially variant-containing fragment when it is determined that the fragment provides a sequence read that includes a sequence of a known cancer variant and that the sequence read's genomic coordinate matches that of the cancer variant. Because sequencing and other processing sometimes introduces errors, there is uncertainty that a fragment sequence showing a cancer mutation actually corresponds to a fragment originating from a cancer cell. There is some chance that a cancer variant-containing sequence read from a fragment is in fact due to sequencing errors instead of an actual somatic mutation.

The term “plurality” refers to more than one element. For example, the term is used herein in reference to a number of nucleic acid molecules or sequence tags that are sufficient to identify significant differences in sequence tags in test samples and qualified samples using the methods disclosed herein. In some embodiments, at least about 3×10⁶ sequence tags of between about 20 and 40 bp are obtained for each test sample. In some embodiments, each test 30×10⁶, 40×10⁶, or 50×10⁶ sequence tags, each sequence tag comprising between about 20 and 40 bp.

The term “paired end reads” refers to reads from paired end sequencing that obtains one read from each end of a nucleic acid fragment. Paired end sequencing may involve fragmenting strands of polynucleotides into short sequences called inserts. Fragmentation is optional or unnecessary for relatively short polynucleotides such as cell free DNA molecules.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules. The term “polynucleotide” includes, without limitation, single- and double-stranded polynucleotide.

The term “test sample” herein refers to a sample, typically derived from a biological fluid, cell, tissue, organ, or organism, comprising a nucleic acid or a mixture of nucleic acids comprising at least one nucleic acid sequence that is to be screened for a biomarker. Such samples include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the assays can be used to detect biomarkers in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “training set” herein refers to a set of training samples that can comprise affected and/or unaffected samples and are used to develop a model for analyzing test samples.

In some embodiments, the training set includes unaffected samples. In these embodiments, thresholds for detecting a biomarker are established using training sets of samples that are unaffected for the biomarker of interest. The unaffected samples in a training set may be used as the qualified samples to identify normalizing sequences, e.g., normalizing chromosomes, and the chromosome doses of unaffected samples are used to set the thresholds for each of the sequences, e.g., chromosomes, of interest. In some embodiments, the training set includes affected samples. The affected samples in a training set can be used to verify that affected test samples can be easily differentiated from unaffected samples.

A training set is also a statistical sample in a population of interest, which statistical sample is not to be confused with a biological sample. A statistical sample often comprises multiple individuals, data of which individuals are used to determine one or more quantitative values of interest generalizable to the population. The statistical sample is a subset of individuals in the population of interest. The individuals may be persons, animals, tissues, cells, other biological samples (i.e., a statistical sample may include multiple biological samples), and other individual entities providing data points for statistical analysis.

Usually, a training set is used in conjunction with a validation set. The term “validation set” is used to refer to a set of individuals in a statistical sample, data of which individuals are used to validate or evaluate the quantitative values of interest determined using a training set. In some embodiments, for instance, a training set provides data for calculating a mask for a reference sequence, while a validation set provides data to evaluate the validity or effectiveness of the mask.

The term “sequence of interest” or “nucleic acid sequence of interest” herein refers to a nucleic acid sequence that is associated with a difference in sequence representation between healthy and diseased individuals. A sequence of interest can be a sequence on a chromosome that is misrepresented, i.e., over- or under-represented, in a disease or genetic condition. A sequence of interest may be a portion of a chromosome, i.e., chromosome segment, or a whole chromosome. For example, a sequence of interest can be a chromosome that is over-represented in an aneuploidy condition, or a gene encoding a tumor-suppressor that is under-represented in a cancer. Sequences of interest include sequences that are over- or under-represented in the total population, or a subpopulation of cells of a subject. A “qualified sequence of interest” is a sequence of interest in a qualified sample. A “test sequence of interest” is a sequence of interest in a test sample.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The terms “threshold value” and “qualified threshold value” herein refer to any number that is used as a cutoff to characterize a sample such as a test sample containing a nucleic acid from an organism suspected of having a medical condition. The threshold may be compared to a parameter value to determine whether a sample giving rise to such parameter value suggests that the organism has the medical condition. In certain embodiments, a qualified threshold value is calculated using a qualifying data set and serves as a limit of diagnosis of a biomarker. If a threshold is exceeded by results obtained from methods disclosed herein, a subject can be diagnosed with a glioblastoma. Appropriate threshold values for the methods described herein can be identified by analyzing normalized values calculated for a training set of samples. Threshold values can be identified using qualified (i.e., unaffected) samples in a training set which comprises both qualified (i.e., unaffected) samples and affected samples. The samples in the training set known to have chromosomal aneuploidies (i.e., the affected samples) can be used to confirm that the chosen thresholds are useful in differentiating affected from unaffected samples in a test set (see the Example herein). The choice of a threshold is dependent on the level of confidence that the user wishes to have to make the classification. In some embodiments, the training set used to identify appropriate threshold values comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or more qualified samples. It may be advantageous to use larger sets of qualified samples to improve the diagnostic utility of the threshold values.

The term “read” refers to a sequence obtained from a portion of a nucleic acid sample. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

The term “genomic read” is used in reference to a read of any segments in the entire genome of an individual.

The term “sequence tag” is herein used interchangeably with the term “mapped sequence tag” to refer to a sequence read that has been specifically assigned, i.e., mapped, to a larger sequence, e.g., a reference genome, by alignment. Mapped sequence tags are uniquely mapped to a reference genome, i.e., they are assigned to a single location to the reference genome.

Unless otherwise specified, tags that map to the same sequence on a reference sequence are counted once. Tags may be provided as data structures or other assemblages of data. In certain embodiments, a tag contains a read sequence and associated information for that read such as the location of the sequence in the genome, e.g., the position on a chromosome. In certain embodiments, the location is specified for a positive strand orientation. A tag may be defined to allow a limited amount of mismatch in aligning to a reference genome. In some embodiments, tags that can be mapped to more than one location on a reference genome, i.e., tags that do not map uniquely, may not be included in the analysis.

As used herein, the terms “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence to which the read or tag maps. For example, if the reference sequence is the whole human genome sequence, an alignment may indicate that a read is present on chromosome 13, and may further indicate that the read is on a particular strand and/or site of chromosome 13.

Aligned reads or tags are one or more sequences that are identified as a match in terms of the order of their nucleic acid molecules to a known sequence from a reference genome.

Alignment can be done manually, although it is typically implemented by a computer algorithm, as it would be impossible to align reads in a reasonable time period for implementing the methods disclosed herein. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. The matching of a sequence read in aligning can be a 100% sequence match or less than 100% (non-perfect match).

The term “mapping” used herein refers to specifically assigning a sequence read to a larger sequence, e.g., a reference genome, by alignment.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. A “genome” refers to the complete genetic information of an organism or virus, expressed in nucleic acid sequences.

In various embodiments, the reference sequence is significantly larger than the reads that are aligned to it. For example, it may be at least about 100 times larger, or at least about 1000 times larger, or at least about 10,000 times larger, or at least about 10⁵ times larger, or at least about 10⁶ times larger, or at least about 10⁷ times larger.

In one example, the reference sequence is that of a full-length human genome. Such sequences may be referred to as genomic reference sequences. In another example, the reference sequence is limited to a specific human chromosome such as chromosome 13. Such sequences may be referred to as chromosome reference sequences. Other examples of reference sequences include genomes of other species, as well as chromosomes, sub-chromosomal regions (such as strands), etc., of any species.

In various embodiments, the reference sequence is a consensus sequence or other combination derived from multiple individuals. However, in certain applications, the reference sequence may be taken from a particular individual.

The term “clinically-relevant sequence” herein refers to a nucleic acid sequence that is known or is suspected to be associated or implicated with a genetic or disease condition.

Determining the absence or presence of a clinically-relevant sequence can be useful in determining a diagnosis or confirming a diagnosis of a medical condition, or providing a prognosis for the development of a disease.

The term “derived” when used in the context of a nucleic acid or a mixture of nucleic acids, herein refers to the means whereby the nucleic acid(s) are obtained from the source from which they originate. For example, in one embodiment, a mixture of nucleic acids that is derived from two different genomes means that the nucleic acids were naturally released by cells through naturally occurring processes such as necrosis or apoptosis. In another embodiment, a mixture of nucleic acids that is derived from two different genomes means that the nucleic acids were extracted from two different types of cells from a subject.

The term “based on” when used in the context of obtaining a specific quantitative value, herein refers to using another quantity as input to calculate the specific quantitative value as an output.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma,” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

As used herein, the term “corresponding to” sometimes refers to a nucleic acid sequence, e.g., a gene or a chromosome, that is present in the genome of different subjects, and which does not necessarily have the same sequence in all genomes, but serves to provide the identity rather than the genetic information of a sequence of interest, e.g., a gene or chromosome.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

As used herein, the term “polynucleotide length” refers to the absolute number of nucleotides in a sequence or in a region of a reference genome. The term “chromosome length” refers to the known length of the chromosome given in base pairs.

The term “subject” herein refers to a human subject as well as a non-human subject such as other mammals. Although the examples herein concern humans and the language is primarily directed to human concerns, the concepts disclosed herein are applicable to genomes from any animal, and are useful in the fields of veterinary medicine, animal sciences, research laboratories and such.

The term “condition” herein refers to “medical condition” as a broad term that includes all diseases and disorders, but can include injuries and normal health situations, such as pregnancy, that might affect a person's health, benefit from medical assistance, or have implications for medical treatments.

The term “sensitivity” as used herein refers to the probability that a test result will be positive when the condition of interest is present. It may be calculated as the number of true positives divided by the sum of true positives and false negatives.

The term “specificity” as used herein refers to the probability that a test result will be negative when the condition of interest is absent. It may be calculated as the number of true negatives divided by the sum of true negatives and false positives.

The term “enrich” herein refers to the process of amplifying polymorphic target nucleic acids contained in a portion of a biological sample and combining the amplified product with the remainder of the biological sample from which the portion was removed. For example, the remainder of the biological sample can be the original biological sample.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

### Sequencing Methods

As indicated above, the prepared samples (e.g., Sequencing Libraries) are sequenced as part of the procedure for identifying a biomarker. Any of a number of sequencing technologies can be utilized.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif), as described below. In addition to the single molecule sequencing performed using sequencing-by-synthesis of Helicos Biosciences, other single molecule sequencing technologies include, but are not limited to, the SMRT.™ technology of Pacific Biosciences, the ION TORREN.™. technology, and nanopore sequencing developed for example, by Oxford Nanopore Technologies.

While the automated Sanger method is considered as a ‘first generation’ technology, Sanger sequencing including the automated Sanger sequencing, can also be employed in the methods described herein. Illustrative sequencing technologies are described in greater detail below.

In one illustrative, but non-limiting, embodiment, the methods described herein comprise obtaining sequence information for the nucleic acids in a test sample (e.g., cellular DNA in a subject being screened for a cancer) using Illumina's sequencing-by-synthesis and reversible terminator-based sequencing chemistry. Template DNA can be genomic DNA, e.g., cellular DNA. In some embodiments, genomic DNA from isolated cells is used as the template, and it is fragmented into lengths of several hundred base pairs. Circulating tumor DNA also exist in short fragments, with a size distribution peaking at about 150-170 bp. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos (not to be confused with the anchor/anchored reads in the analysis of repeat expansion). Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one embodiment, the randomly fragmented genomic DNA is amplified using PCR before it is subjected to cluster amplification. Alternatively, an amplification-free (e.g., PCR free) genomic library preparation is used, and the randomly fragmented genomic DNA is enriched using the cluster amplification alone. The templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adaptors attached to the two ends of the fragment, the adaptors allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced is also referred to as an insert.

In some implementation, a flow cell for clustering in the Illumina platform is a glass slide with lanes. Each lane is a glass channel coated with a lawn of two types of oligos. Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification, a strand folds over, and a second adapter region on a second end of the strand hybridizes with the second type of oligos on the flow cell surface. A polymerase generates a complimentary strand, forming a double-stranded bridge molecule. This double-stranded molecule is denatured resulting in two single-stranded molecules tethered to the flow cell through two different oligos. The process is then repeated multiple times, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

The sequencing by synthesis example described above involves paired end reads, which is used in many of the embodiments of the disclosed methods. Paired end sequencing involves two reads from the two ends of a fragment. When a pair of reads are mapped to a reference sequence, the base-pair distance between the two reads can be determined, which distance can then be used to determine the length of the fragments from which the reads were obtained. In some instances, a fragment straddling two bins would have one of its pair-end read aligned to one bin, and another to an adjacent bin. This gets rarer as the bins get longer or the reads get shorter. Various methods may be used to account for the bin-membership of these fragments. For instance, they can be omitted in determining fragment size frequency of a bin; they can be counted for both of the adjacent bins; they can be assigned to the bin that encompasses the larger number of base pairs of the two bins; or they can be assigned to both bins with a weight related to portion of base pairs in each bin.

Paired end reads may use insert of different length (i.e., different fragment size to be sequenced). As the default meaning in this disclosure, paired end reads are used to refer to reads obtained from various insert lengths. In some instances, to distinguish short-insert paired end reads from long-inserts paired end reads, the latter is also referred to as mate pair reads. In some embodiments involving mate pair reads, two biotin junction adaptors first are attached to two ends of a relatively long insert (e.g., several kb). The biotin junction adaptors then link the two ends of the insert to form a circularized molecule. A sub-fragment encompassing the biotin junction adaptors can then be obtained by further fragmenting the circularized molecule. The sub-fragment including the two ends of the original fragment in opposite sequence order can then be sequenced by the same procedure as for short-insert paired end sequencing described above.

After sequencing of DNA fragments, sequence reads of predetermined length, e.g., 100 bp, are mapped or aligned to a known reference genome. The mapped or aligned reads and their corresponding locations on the reference sequence are also referred to as tags. Sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST, BLITZ (MPsrch), FASTA, BOWTIE, or ELAND (Illumina, Inc., San Diego, Calif., USA). In one embodiment, one end of the clonally expanded copies of the plasma DNA molecules is sequenced and processed by bioinformatics alignment analysis for the Illumina Genome Analyzer, which uses the Efficient Large-Scale Alignment of Nucleotide Databases (ELAND) software.

In one illustrative, but non-limiting, embodiment, the methods described herein comprise obtaining sequence information for the nucleic acids in a test sample, e.g., cellular DNA in a subject being screened for a cancer using single molecule sequencing technology of the Helicos True Single Molecule Sequencing (tSMS) technology (e.g. as described in Harris T. D. et al., Science 320:106-109 [2008]). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments the templates can be at a density of about 100 million templates/cm\hat{ }2. The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

In another illustrative, but non-limiting embodiment, the methods described herein comprise obtaining sequence information for the nucleic acids in the test sample, e.g., cellular DNA in a subject being screened for a cancer, using the 454 sequencing (Roche) (e.g., as described in Margulies, M. et al. Nature 437:376-380 [2005]). 454 sequencing typically involves two steps. In the first step, DNA is sheared into fragments of approximately 300-800 base pairs, and the fragments are blunt-ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (e.g., picoliter-sized wells). Pyrosequencing is performed on each DNA fragment in parallel. Addition of one or more nucleotides generates a light signal that is recorded by a CCD camera in a sequencing instrument. The signal strength is proportional to the number of nucleotides incorporated. Pyrosequencing makes use of pyrophosphate (PPi) which is released upon nucleotide addition. PPi is converted to ATP by ATP sulfurylase in the presence of adenosine 5′ phosphosulfate. Luciferase uses ATP to convert luciferin to oxyluciferin, and this reaction generates light that is measured and analyzed.

In another illustrative, but non-limiting, embodiment, the methods described herein comprises obtaining sequence information for the nucleic acids in the test sample, e.g., cellular DNA in a subject being screened for a cancer, using the SOLiD™ technology (Applied Biosystems). In SOLiD™ sequencing-by-ligation, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide. The sequence can be determined by sequential hybridization and ligation of partially random oligonucleotides with a central determined base (or pair of bases) that is identified by a specific fluorophore. After a color is recorded, the ligated oligonucleotide is cleaved and removed and the process is then repeated.

In another illustrative, but non-limiting, embodiment, the methods described herein comprise obtaining sequence information for the nucleic acids in the test sample, e.g., cellular DNA in a subject being screened for a cancer, using the single molecule, real-time (SMRT™) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phospholinked nucleotides are being incorporated into the growing primer strand. A ZMW detector comprises a confinement structure that enables observation of incorporation of a single nucleotide by DNA polymerase against a background of fluorescent nucleotides that rapidly diffuse in an out of the ZMW (e.g., in microseconds). It typically takes several milliseconds to incorporate a nucleotide into a growing strand. During this time, the fluorescent label is excited and produces a fluorescent signal, and the fluorescent tag is cleaved off. Measurement of the corresponding fluorescence of the dye indicates which base was incorporated. The process is repeated to provide a sequence.

In another illustrative, but non-limiting embodiment, the methods described herein comprise obtaining sequence information for the nucleic acids in the test sample being screened for a cancer, using nanopore sequencing (e.g. as described in Soni G V and Meller A. Clin Chem 53: 1996-2001 [2007]). Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

In another illustrative, but non-limiting, embodiment, the methods described herein comprises obtaining sequence information for the nucleic acids in the test sample in a subject being screened for a cancer, using the chemical-sensitive field effect transistor (chemFET) array (e.g., as described in U.S. Patent Application Publication No. 2009/0026082). In one example of this technique, DNA molecules can be placed into reaction chambers, and the template molecules can be hybridized to a sequencing primer bound to a polymerase. Incorporation of one or more triphosphates into a new nucleic acid strand at the 3′ end of the sequencing primer can be discerned as a change in current by a chemFET. An array can have multiple chemFET sensors. In another example, single nucleic acids can be attached to beads, and the nucleic acids can be amplified on the bead, and the individual beads can be transferred to individual reaction chambers on a chemFET array, with each chamber having a chemFET sensor, and the nucleic acids can be sequenced.

In another embodiment, the DNA sequencing technology is the Ion Torrent single molecule sequencing, which pairs semiconductor technology with a simple sequencing chemistry to directly translate chemically encoded information (A, C, G, T) into digital information (0, 1) on a semiconductor chip. In nature, when a nucleotide is incorporated into a strand of DNA by a polymerase, a hydrogen ion is released as a byproduct. Ion Torrent uses a high-density array of micro-machined wells to perform this biochemical process in a massively parallel way. Each well holds a different DNA molecule. Beneath the wells is an ion-sensitive layer and beneath that an ion sensor. When a nucleotide, for example a C, is added to a DNA template and is then incorporated into a strand of DNA, a hydrogen ion will be released. The charge from that ion will change the pH of the solution, which can be detected by Ion Torrent's ion sensor. The sequencer-essentially the world's smallest solid-state pH meter-calls the base, going directly from chemical information to digital information. The Ion personal Genome Machine (PGM™) sequencer then sequentially floods the chip with one nucleotide after another. If the next nucleotide that floods the chip is not a match. No voltage change will be recorded and no base will be called. If there are two identical bases on the DNA strand, the voltage will be double, and the chip will record two identical bases called. Direct detection allows recordation of nucleotide incorporation in seconds.

In another embodiment, the present method comprises obtaining sequence information for the nucleic acids in the test sample, e.g., a test sample being screened for cancer, using sequencing by hybridization. Sequencing-by-hybridization comprises contacting the plurality of polynucleotide sequences with a plurality of polynucleotide probes, wherein each of the plurality of polynucleotide probes can be optionally tethered to a substrate. The substrate might be flat surface comprising an array of known nucleotide sequences. The pattern of hybridization to the array can be used to determine the polynucleotide sequences present in the sample. In other embodiments, each probe is tethered to a bead, e.g., a magnetic bead or the like. Hybridization to the beads can be determined and used to identify the plurality of polynucleotide sequences within the sample.

In some embodiments of the methods described herein, the mapped sequence tags comprise sequence reads of about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. It is expected that technological advances will enable single-end reads of greater than 500 bp enabling for reads of greater than about 1000 bp when paired end reads are generated. In one embodiment, the mapped sequence tags comprise sequence reads that are 36 bp. Mapping of the sequence tags is achieved by comparing the sequence of the tag with the sequence of the reference to determine the chromosomal origin of the sequenced nucleic acid molecule, and specific genetic sequence information is not needed. A small degree of mismatch (0-2 mismatches per sequence tag) may be allowed to account for minor polymorphisms that may exist between the reference genome and the genomes in the mixed sample.

A plurality of sequence tags is typically obtained per sample. In some embodiments, at least about 3×10⁶ sequence tags, at least about 5×10⁶ sequence tags, at least about 8×10⁶ sequence tags, at least about 10×10⁶ sequence tags, at least about 15×10⁶ sequence tags, at least about 20×10⁶ sequence tags, at least about 30×10⁶ sequence tags, at least about 40×10⁶ sequence tags, or at least about 50×10⁶ sequence tags comprising between 20 and 40 bp reads, e.g., 36 bp, are obtained from mapping the reads to the reference genome per sample. In one embodiment, all the sequence reads are mapped to all regions of the reference genome. In one embodiment, the tags that have been mapped to all regions, e.g., all chromosomes, of the reference genome are analyzed, and the biomarker in the DNA sample is determined.

The accuracy required for correctly determining whether a biomarker is present or absent in a sample, is predicated on the variation of the number of sequence tags that map to the reference genome among samples within a sequencing run (inter-chromosomal variability), and the variation of the number of sequence tags that map to the reference genome in different sequencing runs (inter-sequencing variability). For example, the variations can be particularly pronounced for tags that map to GC-rich or GC-poor reference sequences. Other variations can result from using different protocols for the extraction and purification of the nucleic acids, the preparation of the sequencing libraries, and the use of different sequencing platforms. The present method uses sequence doses (chromosome doses, or segment doses) based on the knowledge of normalizing sequences (normalizing chromosome sequences or normalizing segment sequences), to intrinsically account for the accrued variability stemming from interchromosomal (intra-run), and inter-sequencing (inter-run) and platform-dependent variability. Chromosome doses are based on the knowledge of a normalizing chromosome sequence, which can be composed of a single chromosome, or of two or more chromosomes selected from chromosomes 1-22, X, and Y. Alternatively, normalizing chromosome sequences can be composed of a single chromosome segment, or of two or more segments of one chromosome or of two or more chromosomes. Segment doses are based on the knowledge of a normalizing segment sequence, which can be composed of a single segment of any one chromosome, or of two or more segments of any two or more of chromosomes 1-22, X, and Y.

### Apparatus and System for Determining Variant of Interest

Analysis of the sequencing data and the diagnosis derived therefrom are typically performed using various computer executed algorithms and programs. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel. A processor or group of processors for performing the methods described herein may be of various types including microcontrollers and microprocessors such as programmable devices (e.g., CPLDs and FPGAs) and non-programmable devices such as gate array ASICs or general-purpose microprocessors.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, magnetic media such as disk drives, magnetic tape, optical media such as CDs, magneto-optical media, and hardware devices that are specially configured to store and perform program instructions, such as read-only memory devices (ROM) and random-access memory (RAM).

The computer readable media may be directly controlled by an end-user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include reads and tags derived from a nucleic acid sample, counts or densities of such tags that align with particular regions of a reference sequence (e.g., that align to a chromosome or chromosome segment), reference sequences (including reference sequences providing solely or primarily polymorphisms), chromosome and segment doses, calls such as SNV or aneuploidy calls, normalized chromosome and segment values, pairs of chromosomes or segments and corresponding normalizing chromosomes or segments, counseling recommendations, diagnoses, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

One embodiment provides a computer program product for generating an output indicating the presence or absence of an SNV or aneuploidy associated with a cancer, in a test sample. The computer product may contain instructions for performing any one or more of the above-described methods for determining a chromosomal anomaly. As explained, the computer product may include a non-transitory and/or tangible computer readable medium having a computer executable or compliable logic (e.g., instructions) recorded thereon for enabling a processor to determine if a biomarker call should be made. In one example, the computer product comprises a computer readable medium having a computer executable or compliable logic (e.g., instructions) recorded thereon for enabling a processor to detect a biomarker.

The sequence information from the sample under consideration may be mapped to chromosome reference sequences to identify a number of sequence tags for each of any one or more chromosomes of interest and to identify a number of sequence tags for a normalizing segment sequence for each of said any one or more chromosomes of interest. In various embodiments, the reference sequences are stored in a database such as a relational or object database, for example.

It should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus. Of course, the problem is compounded because reliable biomarker calls generally require mapping thousands (e.g., at least about 10,000) or even millions of reads to one or more chromosomes.

The methods disclosed herein can be performed using a system for evaluation of copy number of a genetic sequence of interest in a test sample. The system comprising: (a) a sequencer for receiving nucleic acids from the test sample providing nucleic acid sequence information from the sample; (b) a processor; and (c) one or more computer-readable storage media having stored thereon instructions for execution on said processor to carry out a method for identifying any biomarker.

In some embodiments, the methods are instructed by a computer-readable medium having stored thereon computer-readable instructions for carrying out a method for identifying any biomarker. Thus, one embodiment provides a computer program product comprising one or more computer-readable non-transitory storage media having stored thereon computer-executable instructions that, when executed by one or more processors of a computer system, cause the computer system to implement a method for evaluation of copy number of a sequence of interest in a test sample comprising normal and tumor cell-free nucleic acids. The method includes: (a) retrieving, by the one or more processors, sequence reads and fragment sizes of DNA fragments obtained from a test sample; (b) assigning, by the one or more processors, the fragments into a plurality of bins representing different fragment sizes; and (c) determining, using the sequence reads and by the one or more processors, an allele frequency of the variant of interest in a prioritized set of bins selected from the plurality of bins, wherein the prioritized set of bins was selected to (i) limit a probability that a quantity of the variant of interest in the prioritized set of bins is below a limit of detection and (ii) increase a probability that a quantity of the variant of interest in the prioritized set of bins is higher than in all bins of the plurality of bins.

In some embodiments, the instructions may further include automatically recording information pertinent to the method such as chromosome doses and the presence or absence of a biomarker in a patient medical record for a human subject providing the biological test sample. The patient medical record may be maintained by, for example, a laboratory, physician's office, a hospital, a health maintenance organization, an insurance company, or a personal medical record website. Further, based on the results of the processor-implemented analysis, the method may further involve prescribing, initiating, and/or altering treatment of a human subject from whom the biological test sample was taken. This may involve performing one or more additional tests or analyses on additional samples taken from the subject.

Disclosed methods can also be performed using a computer processing system which is adapted or configured to perform a method for identifying any biomarker. One embodiment provides a computer processing system which is adapted or configured to perform a method as described herein. In one embodiment, the apparatus comprises a sequencing device adapted or configured for sequencing at least a portion of the nucleic acid molecules in a sample to obtain the type of sequence information described elsewhere herein. The apparatus may also include components for processing the sample. Such components are described elsewhere herein.

Sequence (or other) data can be input into a computer or stored on a computer readable medium either directly or indirectly. In one embodiment, a computer system is directly coupled to a sequencing device that reads and/or analyzes sequences of nucleic acids from samples.

Sequences or other information from such tools are provided via interface in the computer system. Alternatively, the sequences processed by system are provided from a sequence storage source such as a database or other repository. Once available to the processing apparatus, a memory device or mass storage device buffers or stores, at least temporarily, sequences of the nucleic acids. In addition, the memory device may store tag counts for various chromosomes or genomes, etc. The memory may also store various routines and/or programs for analyzing the presenting the sequence or mapped data. Such programs/routines may include programs for performing statistical analyses, etc.

In one example, a user provides a sample into a sequencing apparatus. Data is collected and/or analyzed by the sequencing apparatus which is connected to a computer. Software on the computer allows for data collection and/or analysis. Data can be stored, displayed (via a monitor or other similar device), and/or sent to another location. The computer may be connected to the internet which is used to transmit data to a handheld device utilized by a remote user (e.g., a physician, scientist, or analyst). It is understood that the data can be stored and/or analyzed prior to transmittal. In some embodiments, raw data is collected and sent to a remote user or apparatus that will analyze and/or store the data. Transmittal can occur via the internet, but can also occur via satellite or other connection. Alternately, data can be stored on a computer-readable medium and the medium can be shipped to an end user (e.g., via mail). The remote user can be in the same or a different geographical location including, but not limited to a building, city, state, country, or continent.

In some embodiments, the methods also include collecting data regarding a plurality of polynucleotide sequences (e.g., reads, tags and/or reference chromosome sequences) and sending the data to a computer or other computational system. For example, the computer can be connected to laboratory equipment, e.g., a sample collection apparatus, a nucleotide amplification apparatus, a nucleotide sequencing apparatus, or a hybridization apparatus. The computer can then collect applicable data gathered by the laboratory device. The data can be stored on a computer at any step, e.g., while collected in real time, prior to the sending, during or in conjunction with the sending, or following the sending. The data can be stored on a computer-readable medium that can be extracted from the computer. The data collected or stored can be transmitted from the computer to a remote location, e.g., via a local network or a wide area network such as the internet. At the remote location various operations can be performed on the transmitted data as described below.

Among the types of electronically formatted data that may be stored, transmitted, analyzed, and/or manipulated in systems, apparatus, and methods disclosed herein are the following: Reads obtained by sequencing nucleic acids in a test sample Tags obtained by aligning reads to a reference genome or other reference sequence or sequences. The reference genome or sequence Sequence tag density—Counts or numbers of tags for each of two or more regions (typically chromosomes or chromosome segments) of a reference genome or other reference sequences Identities of normalizing chromosomes or chromosome segments for particular chromosomes or chromosome segments of interest Doses for chromosomes or chromosome segments (or other regions) obtained from chromosomes or segments of interest and corresponding normalizing chromosomes or segments Thresholds for calling chromosome doses as either affected, non-affected, or no call The actual calls of chromosome doses Diagnoses (clinical condition associated with the calls) Recommendations for further tests derived from the calls and/or diagnoses Treatment and/or monitoring plans derived from the calls and/or diagnoses These various types of data may be obtained, stored transmitted, analyzed, and/or manipulated at one or more locations using distinct apparatus. The processing options span a wide spectrum. At one end of the spectrum, all or much of this information is stored and used at the location where the test sample is processed, e.g., a doctor's office or other clinical setting. In other extreme, the sample is obtained at one location, it is processed and optionally sequenced at a different location, reads are aligned and calls are made at one or more different locations, and diagnoses, recommendations, and/or plans are prepared at still another location (which may be a location where the sample was obtained).

In various embodiments, the reads are generated with the sequencing apparatus and then transmitted to a remote site where they are processed to produce calls. At this remote location, as an example, the reads are aligned to a reference sequence to produce tags, which are counted and assigned to chromosomes or segments of interest. Also at the remote location, the counts are converted to doses using associated normalizing chromosomes or segments. Still further, at the remote location, the doses are used to generate calls.

Among the processing operations that may be employed at distinct locations are the following: Sample collection Sample processing preliminary to sequencing Sequencing Analyzing sequence data and deriving biomarker calls Diagnosis Reporting a diagnosis and/or a call to patient or health care provider Developing a plan for further treatment, testing, and/or monitoring Executing the plan Counseling.

Any one or more of these operations may be automated as described elsewhere herein.

Typically, the sequencing and the analyzing of sequence data and deriving biomarker calls will be performed computationally. The other operations may be performed manually or automatically.

Examples of locations where sample collection may be performed include health practitioners' offices, clinics, patients' homes (where a sample collection tool or kit is provided), and mobile health care vehicles. Examples of locations where sample processing prior to sequencing may be performed include health practitioners' offices, clinics, patients' homes (where a sample processing apparatus or kit is provided), mobile health care vehicles, and facilities of biomarker analysis providers. Examples of locations where sequencing may be performed include health practitioners' offices, clinics, health practitioners' offices, clinics, patients' homes (where a sample sequencing apparatus and/or kit is provided), mobile health care vehicles, and facilities of biomarker analysis providers. The location where the sequencing takes place may be provided with a dedicated network connection for transmitting sequence data (typically reads) in an electronic format. Such connection may be wired or wireless and have and may be configured to send the data to a site where the data can be processed and/or aggregated prior to transmission to a processing site. Data aggregators can be maintained by health organizations such as Health Maintenance Organizations (HMOs).

The analyzing and/or deriving operations may be performed at any of the foregoing locations or alternatively at a further remote site dedicated to computation and/or the service of analyzing nucleic acid sequence data. Such locations include for example, clusters such as general-purpose server farms, the facilities of a biomarker analysis service business, and the like.

In some embodiments, the computational apparatus employed to perform the analysis is leased or rented. The computational resources may be part of an internet accessible collection of processors such as processing resources colloquially known as the cloud. In some cases, the computations are performed by a parallel or massively parallel group of processors that are affiliated or unaffiliated with one another. The processing may be accomplished using distributed processing such as cluster computing, grid computing, and the like. In such embodiments, a cluster or grid of computational resources collective form a super virtual computer composed of multiple processors or computers acting together to perform the analysis and/or derivation described herein. These technologies as well as more conventional supercomputers may be employed to process sequence data as described herein. Each is a form of parallel computing that relies on processors or computers. In the case of grid computing these processors (often whole computers) are connected by a network (private, public, or the Internet) by a conventional network protocol such as Ethernet. By contrast, a supercomputer has many processors connected by a local high-speed computer bus.

In certain embodiments, the diagnosis is generated at the same location as the analyzing operation. In other embodiments, it is performed at a different location. In some examples, reporting the diagnosis is performed at the location where the sample was taken, although this need not be the case. Examples of locations where the diagnosis can be generated or reported and/or where developing a plan is performed include health practitioners' offices, clinics, internet sites accessible by computers, and handheld devices such as cell phones, tablets, smart phones, etc. having a wired or wireless connection to a network. Examples of locations where counseling is performed include health practitioners' offices, clinics, internet sites accessible by computers, handheld devices, etc.

In some embodiments, the sample collection, sample processing, and sequencing operations are performed at a first location and the analyzing and deriving operation is performed at a second location. However, in some cases, the sample collection is collected at one location (e.g., a health practitioner's office or clinic) and the sample processing and sequencing is performed at a different location that is optionally the same location where the analyzing and deriving take place.

In various embodiments, a sequence of the above-listed operations may be triggered by a user or entity initiating sample collection, sample processing and/or sequencing. After one or more these operations have begun execution, the other operations may naturally follow. For example, the sequencing operation may cause reads to be automatically collected and sent to a processing apparatus which then conducts, often automatically and possibly without further user intervention, the sequence analysis and derivation biomarker operation. In some implementations, the result of this processing operation is then automatically delivered, possibly with reformatting as a diagnosis, to a system component or entity that processes and reports the information to a health professional and/or patient. As explained such information can also be automatically processed to produce a treatment, testing, and/or monitoring plan, possibly along with counseling information. Thus, initiating an early-stage operation can trigger an end to end sequence in which the health professional, patient, or other concerned party is provided with a diagnosis, a plan, counseling and/or other information useful for acting on a physical condition.

This is accomplished even though parts of the overall system are physically separated and possibly remote from the location of, e.g., the sample and sequence apparatus.

### Glioblastoma-specific Biomarkers and System of Detection

Diagnosis of glioblastoma remains challenging and to-date, tumor markers and imaging characteristics offer only limited sensitivity and specificity. The present inventors have developed systems to more accurately detect the presence of glioblastoma in a patient at an earlier stage, which allows for improved patient outcomes.

In certain aspects of the present invention, a system has been developed that uses tumor extracellular vesicles (EVs) from a patient to detect markers associated with glioblastoma. The system successfully makes this distinction with about 70% to 100% accuracy. In certain aspects, the system successfully makes this distinction with about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or to 100% accuracy.

Briefly, a biological sample is taken from a patient, and target EVs present in the sample are identified to generate a “patient signature.” The patient signature is compared to a “diagnostic panel” of particular biomarkers that correlate with glioblastoma. If at least two or more diagnostic model biomarkers are present in the patient signature, a high-certainty diagnosis is made that the patient has glioblastoma or has a recurrence of glioblastoma. Thus, a high-certainty diagnosis is made with a simple, peripheral blood draw. Based on the diagnosis, appropriate treatment is commenced. This method is useful for diagnosis in patients with a brain tumor, for screening high-risk populations, or in surveillance for glioblastoma recurrence.

### Biological Samples

Since blood contacts most of the tumor, liquid biopsies (LBs) generally involve blood sampling, although other body fluids like mucosa, pleural effusions, urine, and cerebrospinal fluid (CSF) are also analyzed. In certain embodiments, a biological sample is obtained from a patient. In certain embodiments, the sample is a liquid biopsy, such as a blood or plasma sample. In certain embodiments, the sample is mucosa, pleural effusions, urine, and cerebrospinal fluid (CSF). In certain embodiments, the sample contains tumor derived extracellular vesicles (EVs) that are membrane-bound subcellular moieties composed of nucleic acids/proteins.

### Extracellular Vesicles (EVs)

EVs are cell-derived vesicles with a closed double-layer membrane structure. They carry various molecules (proteins, lipids, and RNAs) on their surface as well as in the lumen. Exosomes and other EVs play a critical role in intercellular communication and cellular content transfer, e.g. mRNAs and microRNAs, in both physiological and pathological settings, such as tumor development and progression. The exosomal surface proteins can mediate organ-specific homing of circulating exosomes, and their contents show potential to serve as novel biomarkers, thereby assisting the diagnosis and prognosis prediction of human diseases, such as cancer Approaches to detect and characterize exosomes and other EVs may include: (1) electron microscopy (EM) to assess structure and size; (2) nanoparticle tracking analysis (NTA) to reveal size and zeta potential; (3) protein analysis via immunofluorescence staining, western blotting, ELISA, and mass spectrometry; (4) RNA analysis using array platforms, RNA sequencing, and PCR; and (5) analysis of lipids, sugar, and other components by biochemical assays. Among these approaches, EM provides high-resolution imaging but is neither convenient nor affordable for high throughput molecular profiling of large numbers of circulating exosome samples for potential clinical applications. NTA utilizes light scattering and Brownian motion to measure particle size but does not differentiate between vesicles within a size range of 5× orders of magnitude due to the low dynamic range of the camera. In addition, NTA is not suitable for molecular profiling of exosomes because of low sensitivity to fluorescent signals.

### Non-Blood Liquid

In addition to circulatory fluids like plasma or serum, other body fluids such as saliva and urine can be used as liquid biopsies. Saliva offers practical advantages with regard to ease of access, non-invasiveness, and cost effectiveness in sampling, even more so than plasma or serum. Novel electrochemical sensor-based technologies like an electric field-induced release and measurement (EFIRM) have been shown to detect EGFR mutations (tyrosine kinase domain) from bodily fluids like saliva in patients. Similar EFIRM based technologies have been used in developing salivary biomarkers.

The completely non-invasive nature of urine sampling, relative to tissue or even blood, makes it a quite useful candidate in LBs, particularly in cases where repeated sampling is required to monitor tumor progression and therapeutic outcomes.

### Probes, Diagnostic Panels, and Kits

In certain embodiments, the present invention provides a diagnostic panel of probes that hybridize to nucleic acid. In certain embodiments the nucleic acid is ctDNA.

In certain embodiments, the panel comprises at least 1, 2, 3, 4, 5, 6, 7, 8, 9, 10, 11, 12, 13, 14, 15, 16, 17, 18, 19, 20, 21, 22, 23, or 24 IDH1WT biomarker probes listed in Table 1. In certain embodiments, the panel comprises two or more biomarker probes listed in Table 1. In certain embodiments, the panel comprises three or more biomarker probes listed in Table 1.

In certain embodiments, the panel comprises at least 1, 2, 3, 4, 5, 6, 7, 8, 9, 10, 11, 12, 13, 14 or 15 TP53mut biomarker probes listed in Table 2. In certain embodiments, the panel comprises two or more biomarker probes listed in Table 2. In certain embodiments, the panel comprises three or more biomarker probes listed in Table 2.

In certain embodiments, the panel comprises at least 1, 2, 3, 4, 5, 6, 7, 8, 9, 10, 11, 12, 13, 14, 15, 16, 17, 18, 19, or 20 TERTmut biomarker probes listed in Table 3. In certain embodiments, the panel comprises two or more biomarker probes listed in Table 3. In certain embodiments, the panel comprises three or more biomarker probes listed in Table 3.

In certain embodiments, the panel comprises at least 1, 2, 3, 4, 5, 6, 7, 8, 9, 10, 11, 12, 13, 14, 15, 16, or 17 MGMTmeth biomarker probes listed in Table 4. In certain embodiments, the panel comprises two or more biomarker probes listed in Table 4. In certain embodiments, the panel comprises three or more biomarker probes listed in Table 4. In certain embodiments, each probe comprises a unique label.

One aspect provides a kit comprising a collection of probes, wherein the collection comprises a panel of probes, and instructions for use in analyzing a biological sample. In certain embodiments, the panel comprises at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1).

In certain embodiments, the panel comprises at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8, and AC008964.1 (Table 2).

In certain embodiments, the panel comprises at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4).

In certain embodiments, the panel comprises at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2 and LARGE-IT1 (Table 3). In certain embodiments, the biological sample is a liquid biopsy. In certain embodiments, the liquid biopsy is blood or a blood product.

Methods of Detecting Biomarkers Specific for Glioblastoma Currently, patients are getting MRI scans and going through highly invasive and expensive surgery to obtain biopsy samples for diagnosis. The present technology provides a non-invasive, cost-effective cancer screening tool.

One aspect provides a method of detecting the presence of biomarkers associated with an increased risk of glioblastoma in a human subject, comprising:
(i) contacting RNA/DNA from a biological sample containing cells from the subject with the panel of probes to form hybridized target sequences;
(ii) detecting the hybridized target sequences;
(iii) determining the number of hybridized target sequences detected; and
(iv) indicating that the human subject has an increased risk of glioblastoma if more than 2 biomarkers are present. In certain embodiments, the panel comprises at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1). In certain embodiments, the panel comprises at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8 and AC008964.1 (Table 2). In certain embodiments, the panel comprises at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4). In certain embodiments, the panel comprises at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1 (Table 3).

In certain embodiments, the biological sample is a liquid biopsy.

In certain embodiments, the liquid biopsy is blood or a blood product.

In certain embodiments, the biological sample is subdivided into individual subsamples, and a different single probe is applied to each subsample.

One aspect provides a method of detecting glioblastoma in a patient comprising: (a) obtaining a sample from the patient;
(b) isolating extracellular vesicles from the sample; and
(c) detecting expression of at least one cancer marker in the isolated extracellular vesicles.

In certain embodiments, step (b) extracellular vesicles are isolated by ultracentrifugation.

In certain embodiments, the extracellular vesicles are exosomes.

In certain embodiments, the extracellular vesicles are about 30 to about 150 nm in size.

In certain embodiments, the extracellular vesicles express at least one of the exosomal markers IDH1-wt or IDH1 R132H, a p53 mutation, MGMT methylation, or TERT promoter mutation.

In certain embodiments, in step (c), expression of the at least one cancer marker is detected using a micro flow cytometer.

In certain embodiments, step (c) comprises determining a differential expression profile for at least one cancer marker in the sample as compared to a control.

In certain embodiments, the markers on individual extracellular vesicles within the isolated extracellular vesicles are detected.

In certain embodiments, the sample is a body fluid sample or a tissue sample.

Once the patient signature is determined for the biomarker found in the sample and compared to the Glioblastoma “diagnostic panel” appropriate treatment is commenced. Patients go under surgery followed by IR and Chemotherapy treatment.

One aspect provides a method of treating a human subject for glioblastoma, comprising: (i) contacting DNA from a biological sample containing cells from the subject with the panel of probes to form hybridized target sequences;
(ii) detecting the hybridized target sequences;
(iii) determining the number of hybridized target sequences detected;
(iv) indicating that the human subject has an increased risk of glioblastoma if more than 2 biomarkers are present; and
(v) administering an appropriate treatment to the patient.

In certain embodiments, the panel comprises at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1 (Table 1). In certain embodiments, the panel comprises at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8 and AC008964.1 (Table 2). In certain embodiments, the panel comprises at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3 (Table 4). In certain embodiments, the panel comprises at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1 (Table 3).

In certain embodiments, the panel comprises at least 2 probes.

In certain embodiments, the panel comprises at least 3 probes.

In certain embodiments, the treatment comprises IR and chemotherapy.

The invention will now be illustrated by the following non-limiting Example.

### Example 1

Extracellular vesicle-based cancer panels diagnose glioblastomas with high sensitivity and specificity Glioblastoma is one of the most devastating neoplasms of the central nervous system.

This study focused on the development of serum extracellular vesicle (EV)-based glioblastoma tumor marker panels that can be used in the clinic to diagnose glioblastomas and to monitor tumor burden, progression, and regression in response to treatment. RNA sequencing studies were performed using RNA isolated from serum EVs of both patient (n=85) and control donors (n=31). RNA sequencing results for preoperative glioblastoma EVs compared to control EVs revealed 569 differentially expressed genes (DEGs, 2XFC, FDR<0.05). Using these DEGs, we developed serum EV-based biomarker panels for the following: wild-type IDH1 status (96% sensitivity/80% specificity), MGMT promoter methylation (91% sensitivity/73% specificity), mutation in the p53 gene (100% sensitivity/89% specificity), and TERT promoter mutation (89% sensitivity/100% specificity). To our knowledge, this is the first study showing that serum EV-based biomarker panels can diagnose glioblastomas with high sensitivity and specificity.

Glioblastoma, which includes World Health Organization (WHO) tumors of the central nervous system (CNS) grade 4, is the most common malignant primary brain tumor in adults, with a median overall survival of only 16-18 months after diagnosis. Glioblastomas comprise a highly malignant group of tumors that commonly occur in elderly patients (median age at diagnosis, 65 years). Historically, the histopathological diagnosis of glioblastoma was primarily based on the presence of necrosis and/or microvascular proliferation in addition to anaplastic features such as prominent cellular and nuclear atypia, frequent mitotic figures, areas of necrosis, and vascular proliferation. However, in the most recent WHO CNS 2021 classification, the presence of epidermal growth factor receptor (EGFR) amplification and/or whole chromosome 7 gain and whole chromosome 10 loss (+7/−10) and/or telomerase reverse transcriptase (TERT) promoter mutations indicate a molecular diagnosis of glioblastoma, CNS WHO grade 4, in isocitrate dehydrogenase (IDH)-wt astrocytic gliomas.

The WHO 2016 classification of CNS tumors defines specific glioma entities based on a molecular signature to create optimized prognosis and treatment stratification categories. The most critical molecular signature for optimized categories for prognosis and treatment stratification of diffuse gliomas is IDH mutation according to the WHO 2016 classification, and IDH-wt gliomas have a worse prognosis than IDH-mutant gliomas. In addition to these genetic changes, p53 mutation is the most frequent and earliest detectable genetic alteration and already present in 60% of precursor low-grade astrocytomas; moreover, accumulation of other mutations leads to malignant progression of glioblastoma over time.

Clinical management of glioblastoma suffers from a lack of early diagnostic tests. These tumors are mainly diagnosed through resection/biopsy of the tumor followed by neuroimaging techniques (e.g., magnetic resonance imaging-MRI). Although surgery is the gold standard for diagnosis, this approach only provides a temporal and spatial snapshot of these heterogeneous tumors that display longitudinal changes due to the selective pressures of ongoing therapy. Furthermore, treatment efficacy is difficult to determine in many cases, particularly during early therapy, because therapy-associated tissue inflammation often resembles the effects of disease progression termed pseudoprogression by MRI. In addition to pseudoprogression, it can be challenging to differentiate radionecrosis, tumor progression, or pseudoresponse through MRI or positron emission tomography (PET) scans. As a result, determining actual progression of the disease is difficult, which impacts timely response to treatment failure in patients. This issue has been a challenge in clinical trials, for which no reliable surrogate marker is available for disease monitoring. Thus, a noninvasive, longitudinal strategy would be instrumental for many purposes, such as diagnosis, prognostic assessment, prediction of treatment response, and assessment of tumor progression, as tissue-based tumor profiles are subject to sampling bias, providing only a snapshot of tumor heterogeneity, and cannot be obtained repeatedly. These limitations indicate an unmet need for noninvasive, practical, and flexible approaches for clinical monitoring of glioblastoma. Circulating biomarkers are an appealing potential solution.

Therefore, patient serum-derived extracellular vesicle (EV)-glioblastoma tumor marker panels were developed that can be implemented in the clinic to diagnose glioblastoma and to monitor tumor burden and progression as well as tumor response to therapy.

EVs are membrane-bound nanovesicles consisting of exosomes (50-200 nm), microvesicles (>100-1 μm), apoptotic bodies (50-2 μm), and large oncosomes (>1 μm) and are actively released by both healthy cells and cancer cells. EVs are present in biological fluids and involved in multiple physiological and pathological processes. EVs consist of a lipid bilayer that contains both transmembrane and nonmembrane proteins and miRNAs, mRNAs, and either single- or double-stranded DNA. After docking onto a cell, they deliver cargo and thereby alter molecular activities in the recipient cells. EVs play a critical role in the intercellular communication between distant cells and participate in several pathological processes of tumor cells, including proliferation, migration, invasion, and angiogenesis. The accessibility of EVs in biofluids, such as cerebrospinal fluid (CSF) and blood, provides new diagnostic opportunities for minimally invasive biomarker discovery for glioblastoma. Recent studies have shown the use of miRNA and mRNA content of EVs as a potential diagnostic tool that can also be used in genetic subtyping. EVs from glioma patients have been shown to be a source for detection of clinically relevant prognostic biomarkers, such as IDH1-R132H and EGFRvIII, and have been successfully extracted from blood and CSF. Additionally, a higher EV concentration in the plasma of glioblastoma patients than in healthy individuals was demonstrated and linked to tumor recurrence after resection. Overall, EVs are a potential biomarker to diagnose glioblastoma, monitor disease progression, and distinguish patients with tumors from both healthy controls and patients harboring other brain lesions.

In this study, RNA sequencing of serum EVs isolated from a large cohort of glioblastoma patients and age-matched cancer-free healthy controls was applied to discover new tumor markers with diagnostic and monitoring utility. The data obtained were used to develop cancer panels that could distinguish patients with IDH-wt glioblastoma from controls and predict TERT promoter mutation, MGMT methylation status, and p53 mutation with high sensitivity and specificity.

### Results

### Demographic and Clinical Features of the Study Cohort and Transcriptome Analyses of Glioblastoma Patient Serum-Derived EVs

A study was designed comprising a cohort of 91 glioblastoma patients (52 male, 39 female) and 31 control subjects (14 male, 17 female). All subjects in this cohort were glioblastoma patients who were newly diagnosed and received no prior treatment or surgery. All patients were on preoperative dexamethasone (4 mg QID) and antiepileptic agents (levetiracetam 500 mg BID). Age- and sex-matched subjects of the healthy control group were free of any malignancies or systemic diseases. Blood samples were collected after overnight fasting, and sera were isolated and stored at −80° C. according to the Institutional Review Board protocols approved by Hacettepe University Faculty of Medicine. The demographic and clinical features of the patients are summarized in Table 5.

TABLE 5
Table 5. Demographic and clinical features of patients

| Controls | Male | | Female | | Total # | MGMT status | Male | Female | Total # |
|---|---|---|---|---|---|---|---|---|---|
| Number of patients | 14 | | 17 | | 31 | Unmethylated | 22 | 15 | 37 |
| Age, years, | 51.5 | (27-86) | 47 | (29-61) | | Methylated | 24 | 18 | 42 |
| median (min-max) | | | | | | NA | 6 | 6 | 12 |
| Glioblastoma | | | | | | TERT promoter status C228T | | | |
| Number of patients | 52 | | 39 | | 91 | Wild-type | 11 | 11 | 22 |
| Age, years, | 55.02 | (24-84) | 54.44 | (24-81) | | Mutant | 35 | 22 | 57 |
| median (min-max) | | | | | | NA | 6 | 6 | 12 |
| IDH1 status (R132H) | | | | | | ATRX status | | | |
| Wild-type | 48 | | 37 | | 85 | Wild-type | 18 | 20 | 38 |
| Mutant | 4 | | 2 | | 6 | Mutant | 1 | 3 | 4 |
| NA | | | | | | NA | 33 | 16 | 49 |

| IDH2 status (R172H) | | | | BRAF status (600) | | | |
|---|---|---|---|---|---|---|---|
| Wild-type | 52 | 39 | 79 | Wild-type | 46 | 33 | 79 |
| Mutant | 0 | 0 | 0 | Mutant | 0 | 0 | 0 |
| | | | | NA | 6 | 6 | 12 |
| TP53 status | | | | H3F3A status (27/34) | | | |
| Wild-type | 31 | 24 | 55 | Wild-type | 46 | 33 | 79 |
| Mutant | 21 | 15 | 36 | Mutant | 0 | 0 | 0 |
| NA | 0 | 0 | 0 | NA | 6 | 6 | 12 |

The status of IDH1, IDH2, p53, BRAF, and H3F3A mutations, MGMT promoter methylation, and TERT promoter C228T mutation of tumor specimens, were analyzed as described in the methods below. Portions of the IDH1 and IDH2 genes were amplified, spanning codon 132 (IDH1) and codon 172 (IDH2) mutation sites, respectively. It was found that 85 of 91 patients carried IDH1-wt; the IDH1 R132H mutation was found in only 6 patients. IDH2 mutation was not detected in the samples. p53 IHC staining of the samples was performed and p53 mutation was found in 36 of 91 (≥50% nuclear staining was considered mutated p53). MGMT promoter methylation was observed in 42 of 79 patients and TERT promoter mutation in 57 of 79 patients. ATRX mutation was detected in 4 of 42 patients. Mutation in BRAF or H3F3A was not in the cohort (Table 5).

### RNA-Sequencing of Circulating EVs

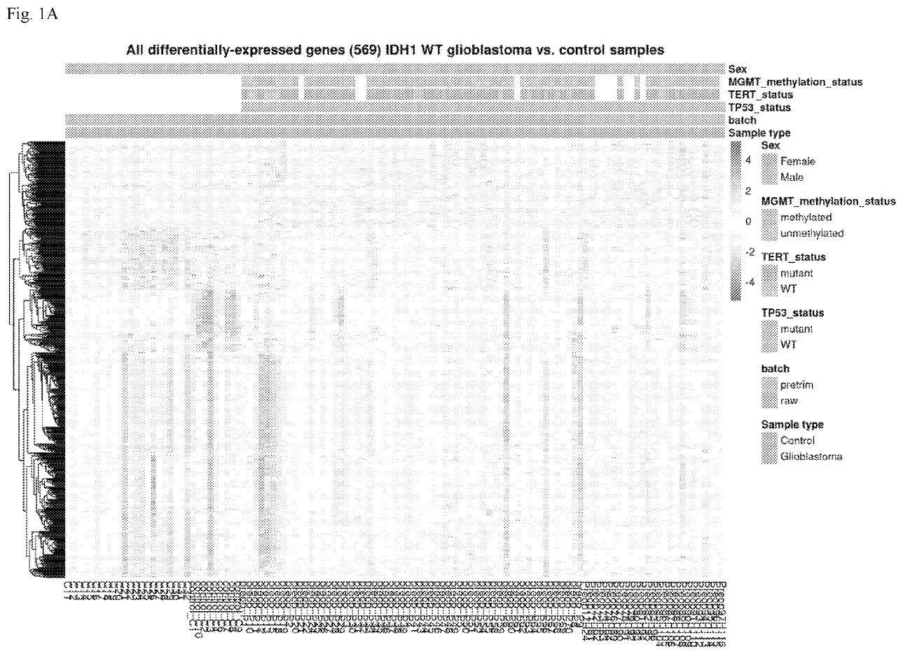

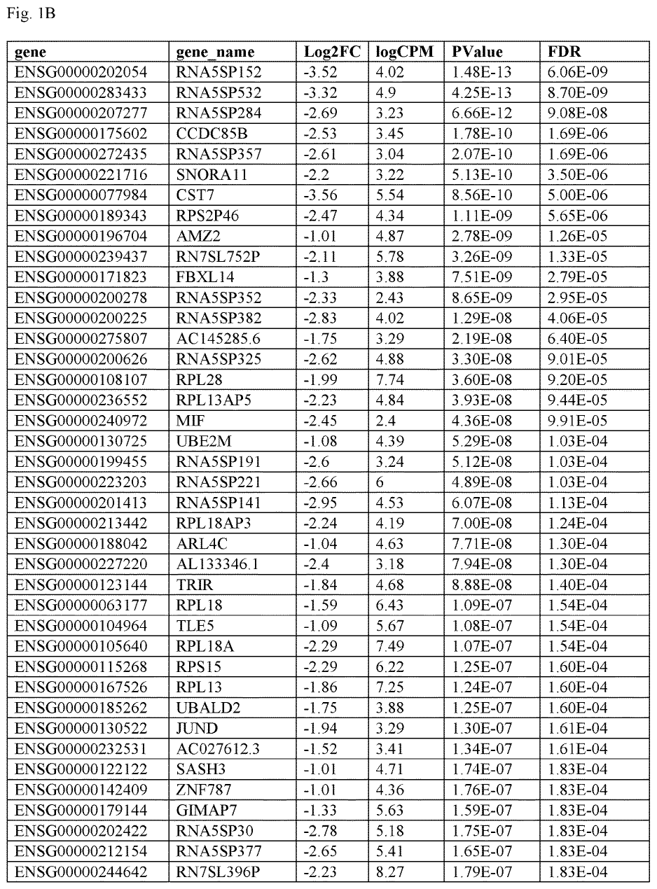

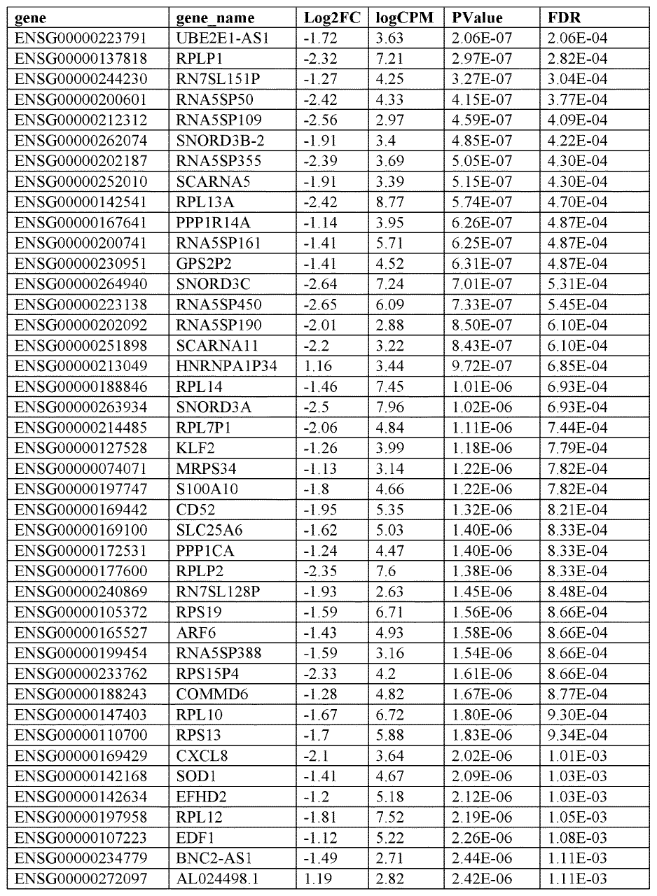

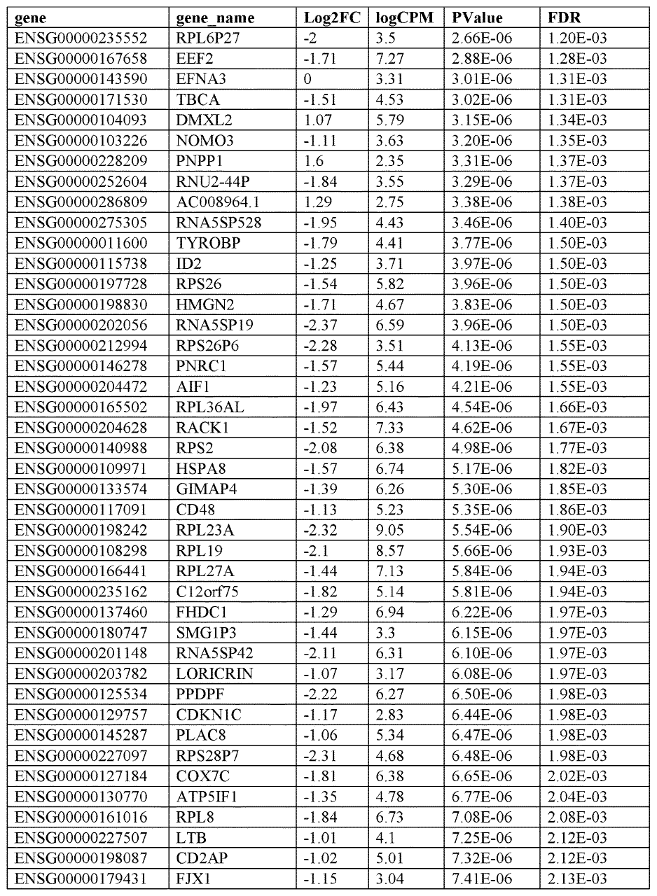

To identify biological markers that can be used in minimally invasive approaches to diagnose and monitor glioblastoma, a membrane-based affinity binding protocol was used to isolate EVs from serum samples of 91 glioblastoma patients and 31 age- and sex-matched control subjects and performed total RNA-seq on EVs. EVs were characterized by nanoparticle tracking analysis (NTA) and western blotting using antibodies against three EV-specific markers, CD63, TSG101, and CD81, according to the 2018 International Society for Extracellular Vesicles (ISEV) guidelines. First, isolated EVs were isolated from patient serum and compared to control subject EVs, and a significant increase was found in CD63 protein levels as an EV marker in glioblastoma patient sera compared to control EVs. Preoperative glioblastoma and control samples revealed 569 differentially expressed genes (DEGs), with an absolute fold-change of 2 and an FDR-adjusted p value of less than 0.05 (FIGS. 1A-1B). A heatmap of the 569 DE genes (protein-coding and noncoding) was generated. Overall, 309 genes of 569 are protein-coding. Among these 309 genes, nine were upregulated and 300 downregulated in glioblastoma patient serum-derived EVs compared to control subject serum EVs.

Serum-Derived EVs from Patients with IDH1-Wt Glioblastoma have Distinct Transcriptomic Features

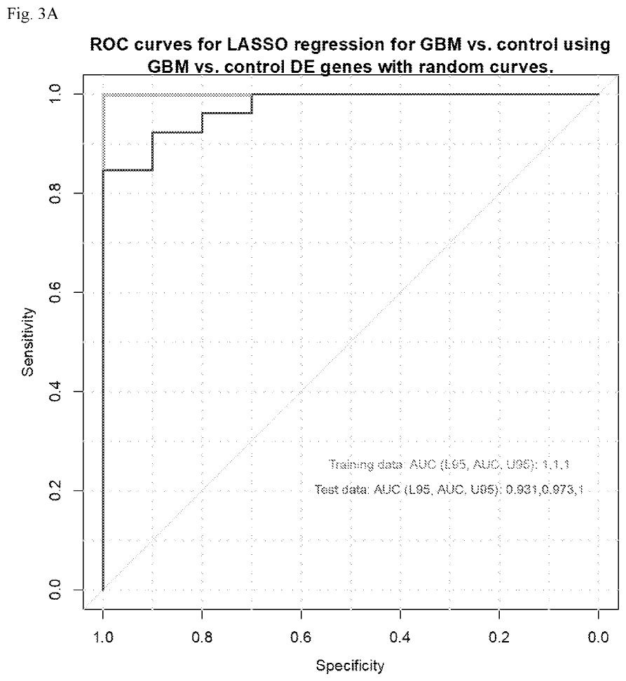

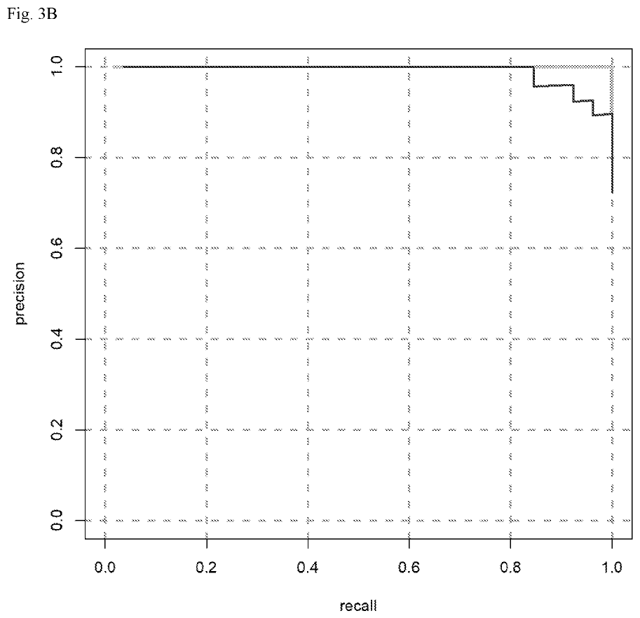

To identify a gene signature to classify control subjects and patients with glioblastoma (i.e., IDH1 wild-type), we used 569 genes differentially expressed between the two groups as predictors of diagnosis (0=control, 1=glioblastoma). We separated the normalized (counts per million) expression data into a training set (70% of samples) and testing set (30% of samples) and used LASSO regression to develop a predictive model. The best predictors were used to create the final model and receiver operating characteristic (ROC) curve analysis to assess the performance of the final model. This analysis resulted in 96% sensitivity and 80% specificity, with 24 dysregulated genes of 569 in IDH-wt glioblastoma (FIGS. 3A-3B, and Table 1).

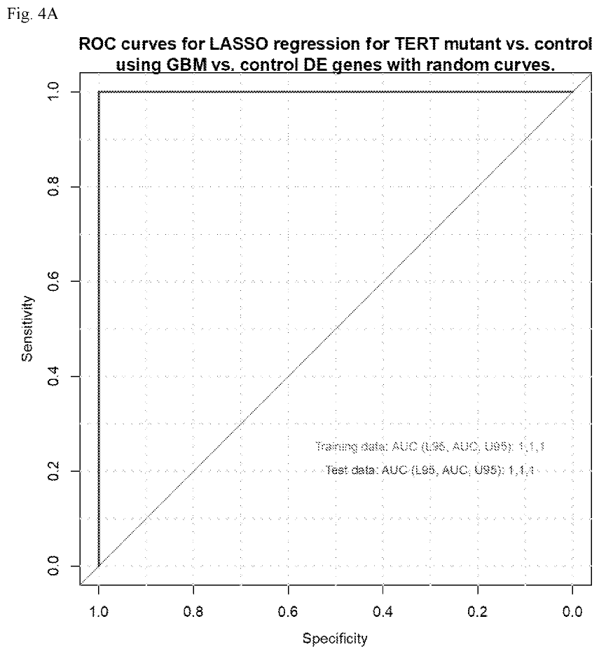

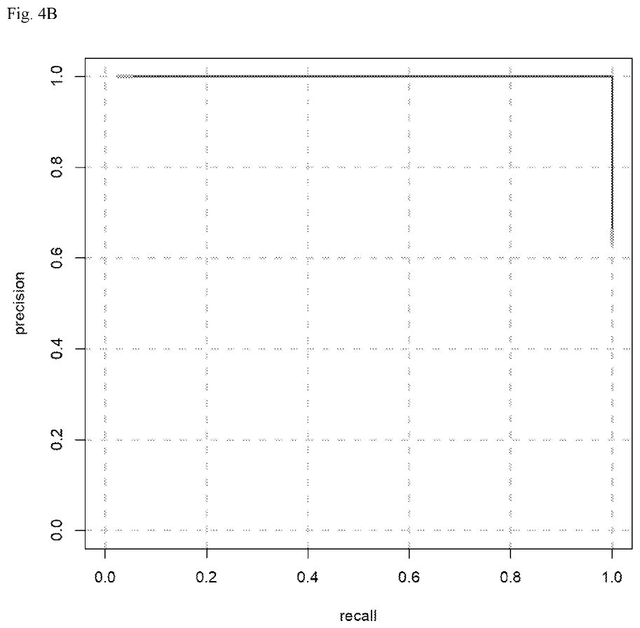

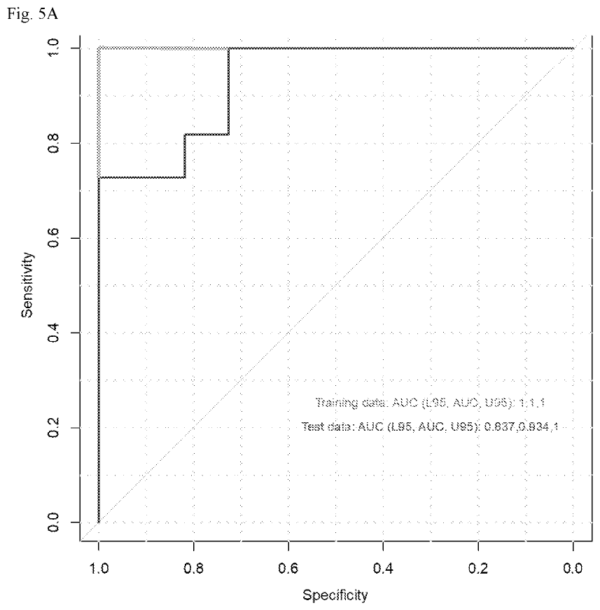

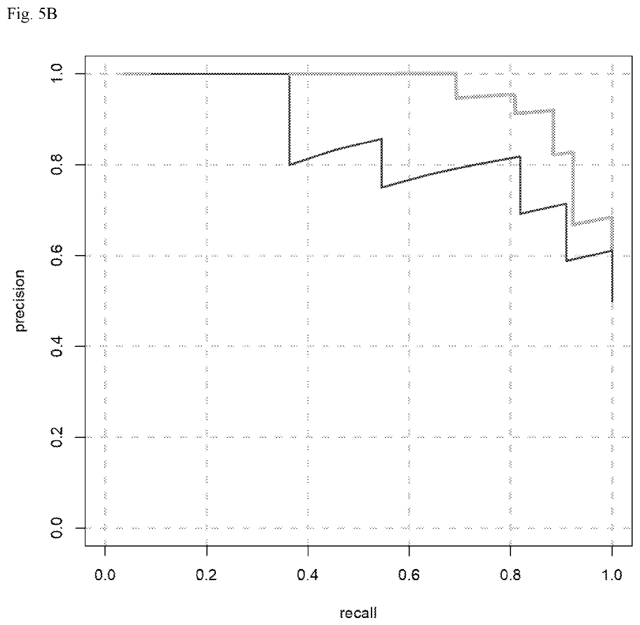

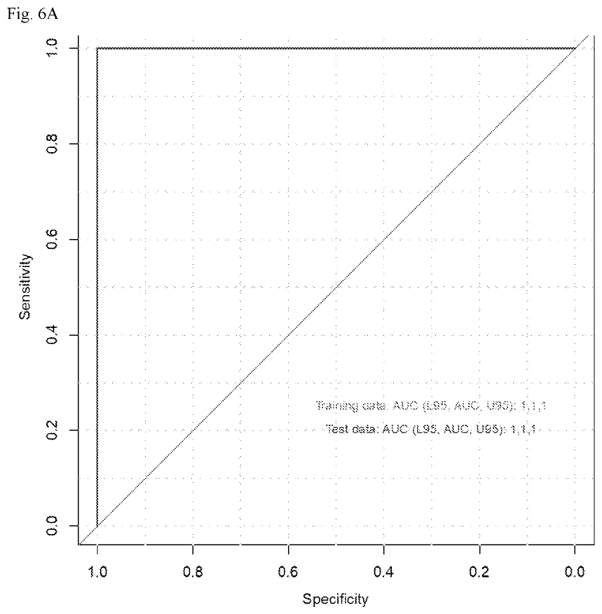

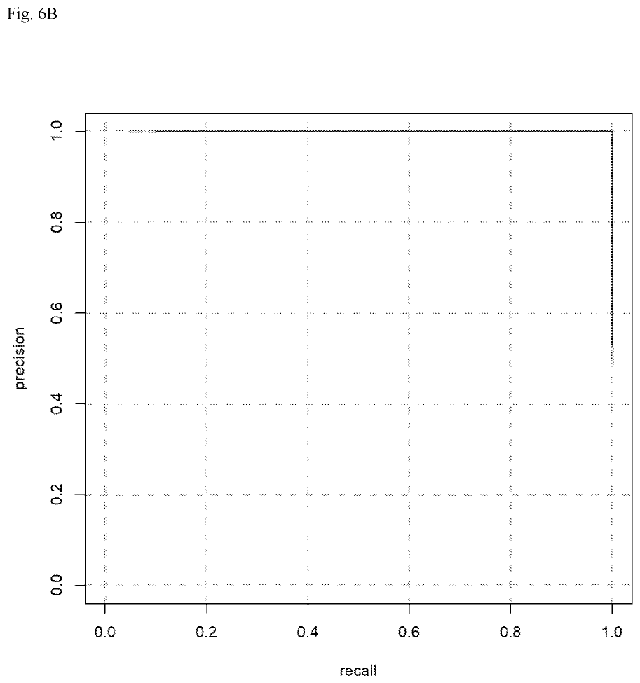

Similar regression analyses were also performed for TERT promoter mutation (Table 3). and MGMT promoter methylation status as illustrated in FIGS. 5A-5B. The TERT promoter mutation cancer panel with 20 different sets of genes showed 89% sensitivity and 100% specificity (FIGS. 4A-4B and Table 3). The MGMT promoter methylation panel showed 91% sensitivity and 73% specificity, with 17 of 569 genes (FIGS. 5A-5B and Table 4). Moreover, the p53 gene mutation cancer panel with 15 genes of 569 showed 100% sensitivity and 89% specificity (FIGS. 6A-6B and Table 2).

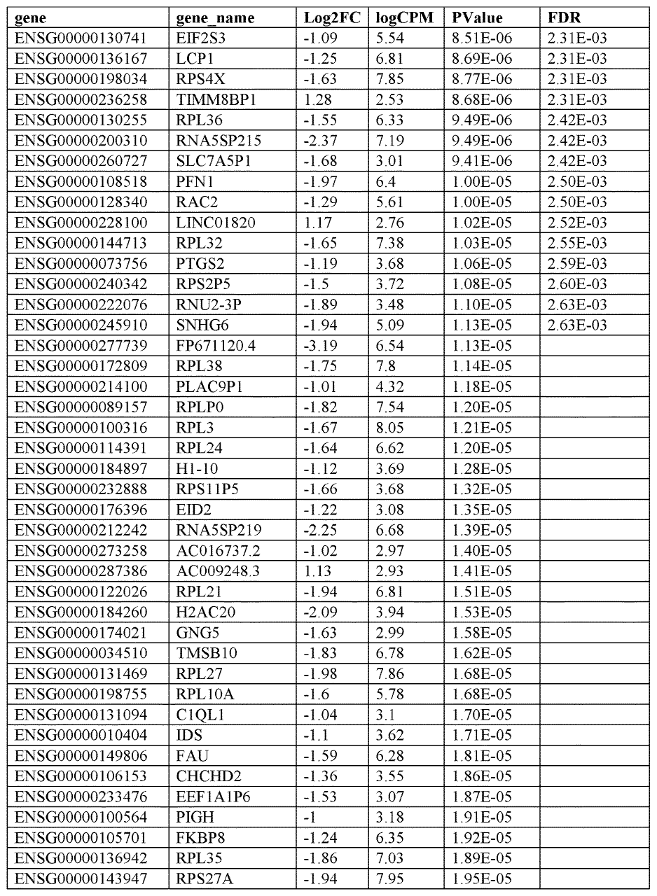

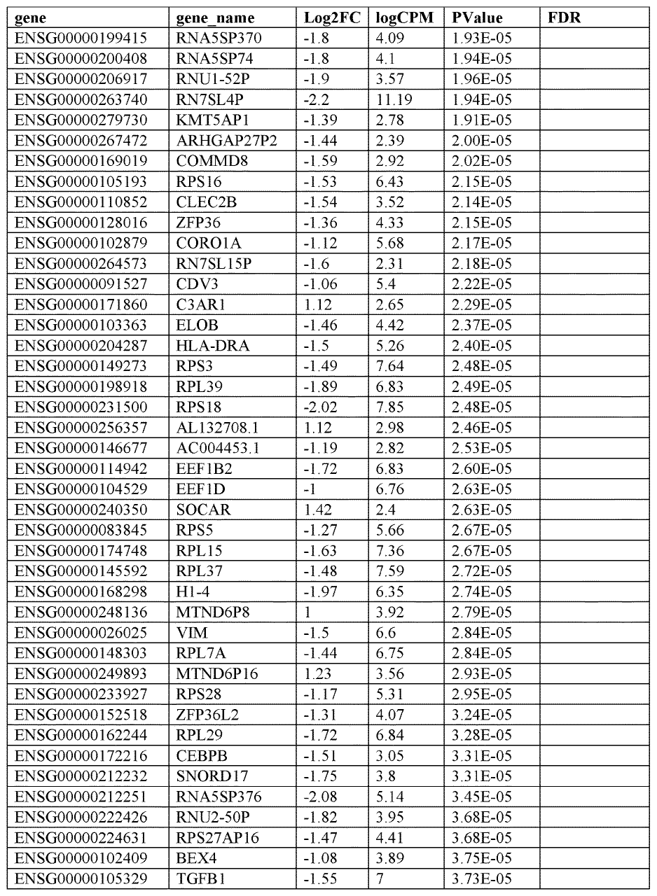

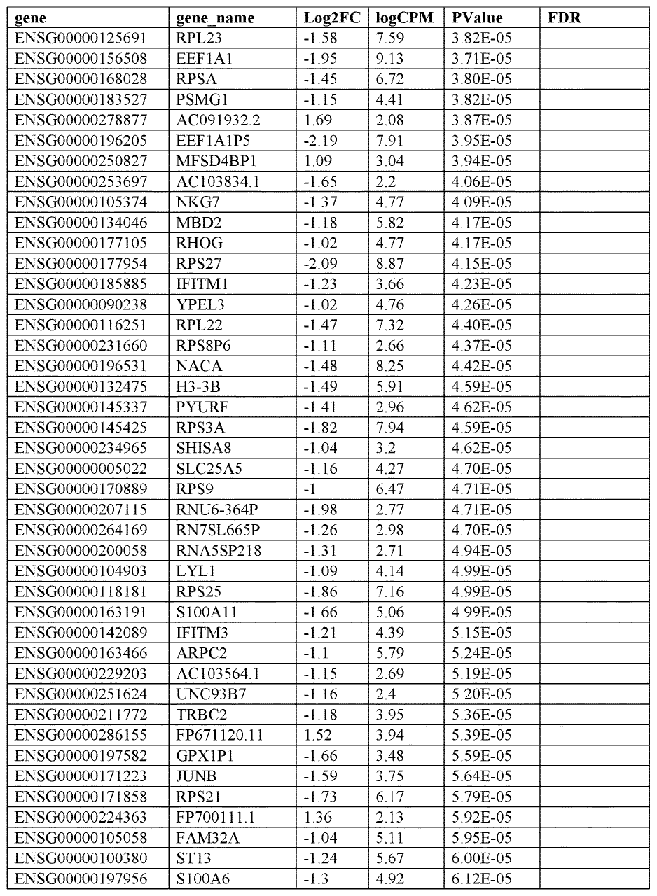

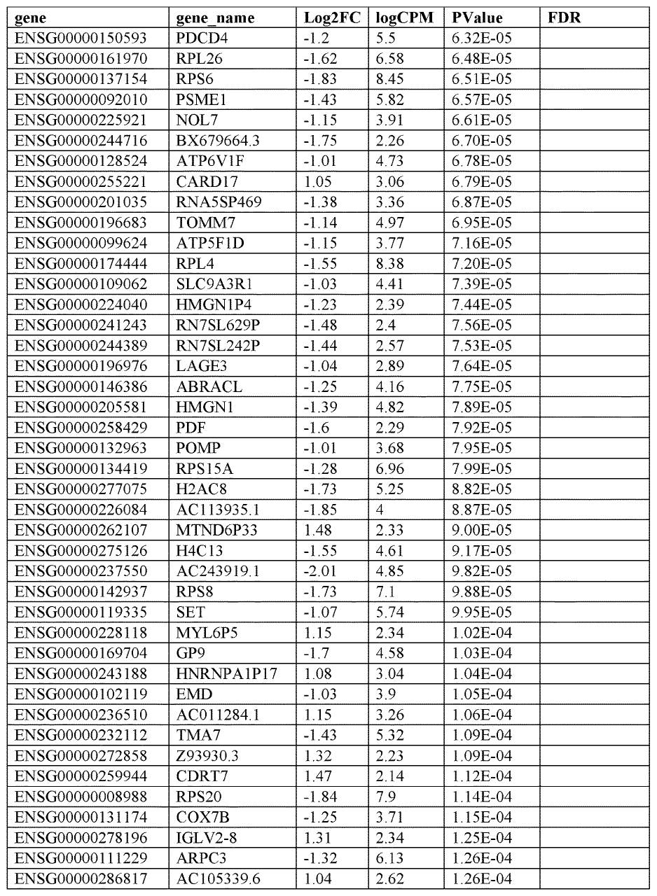

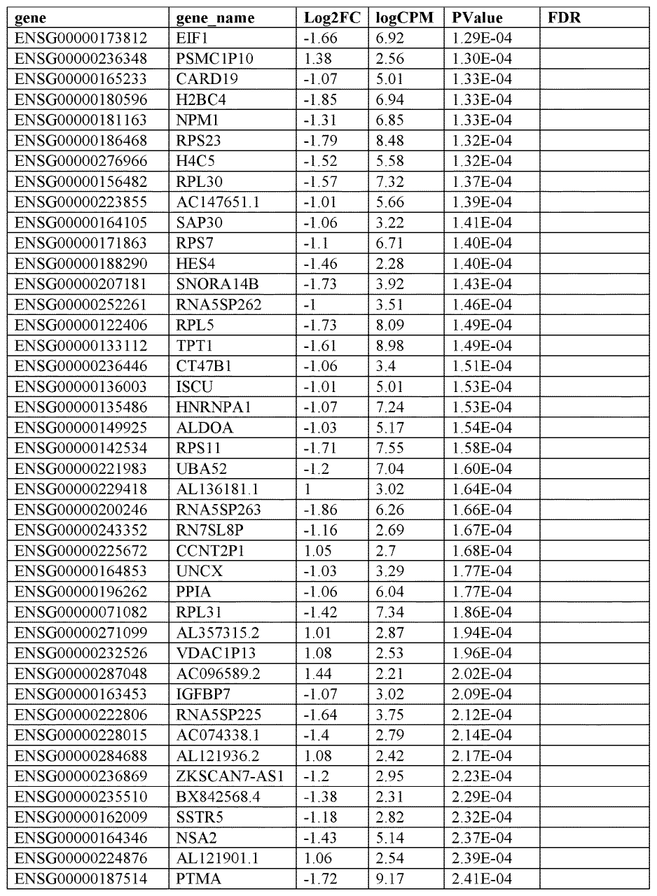

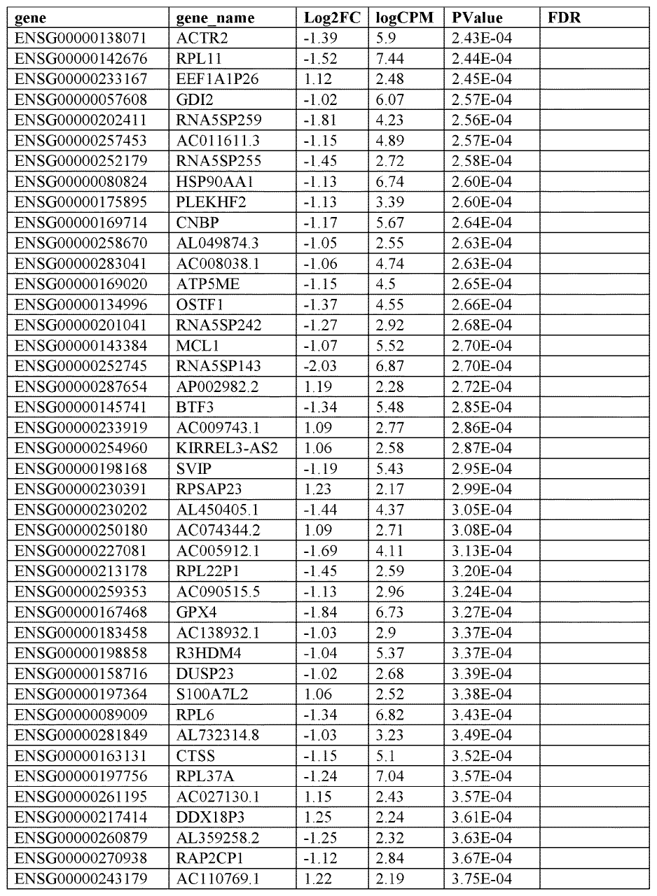

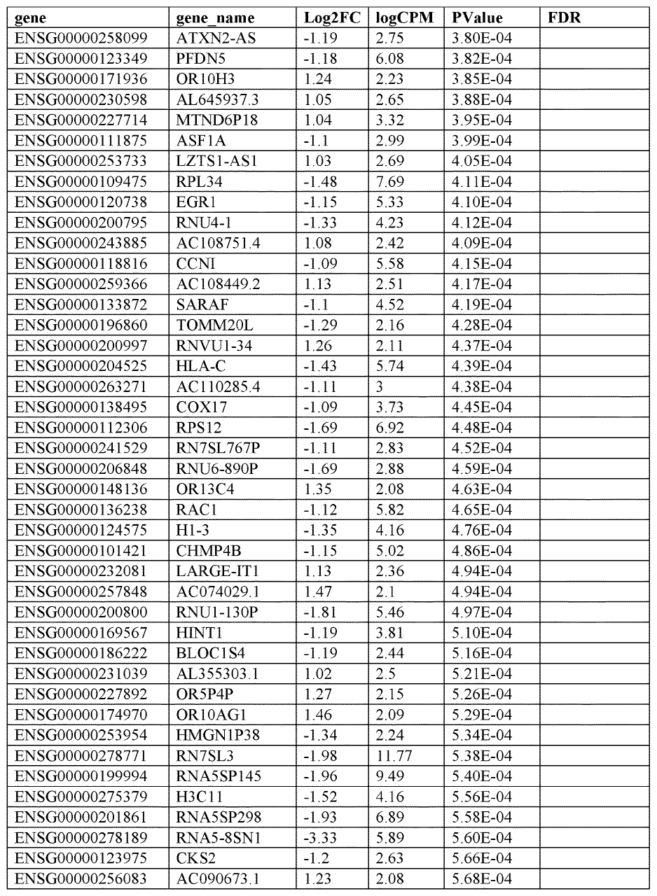

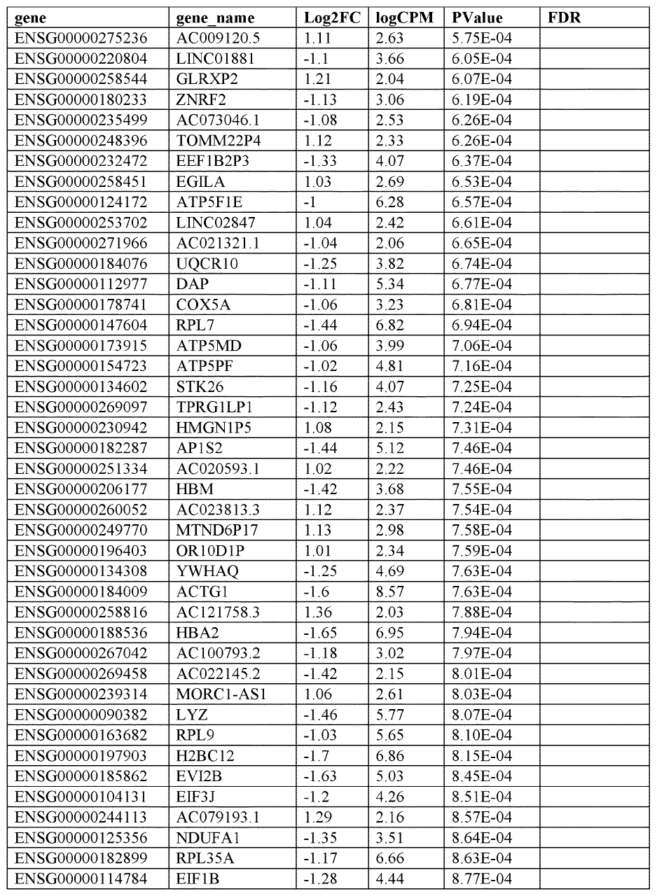

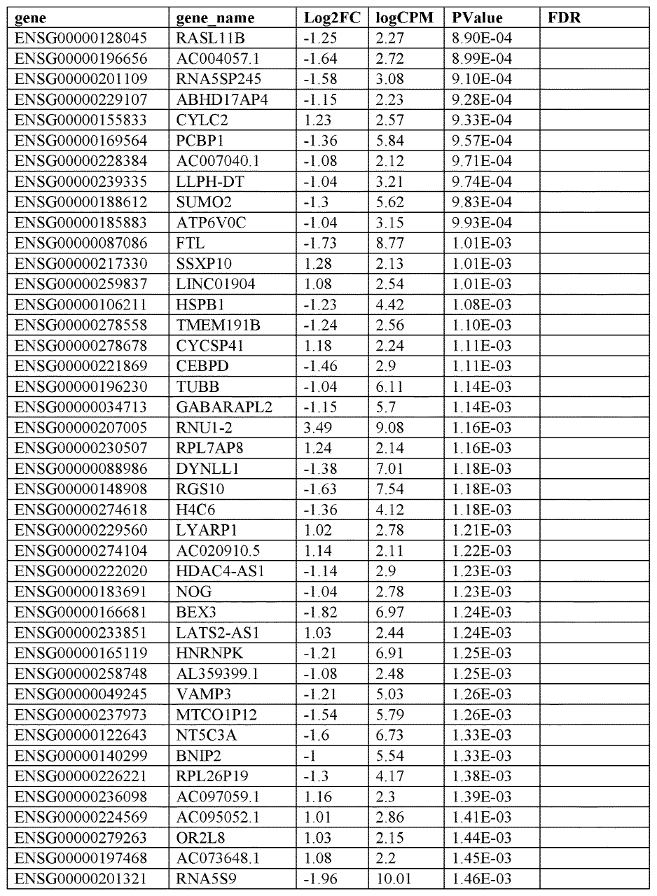

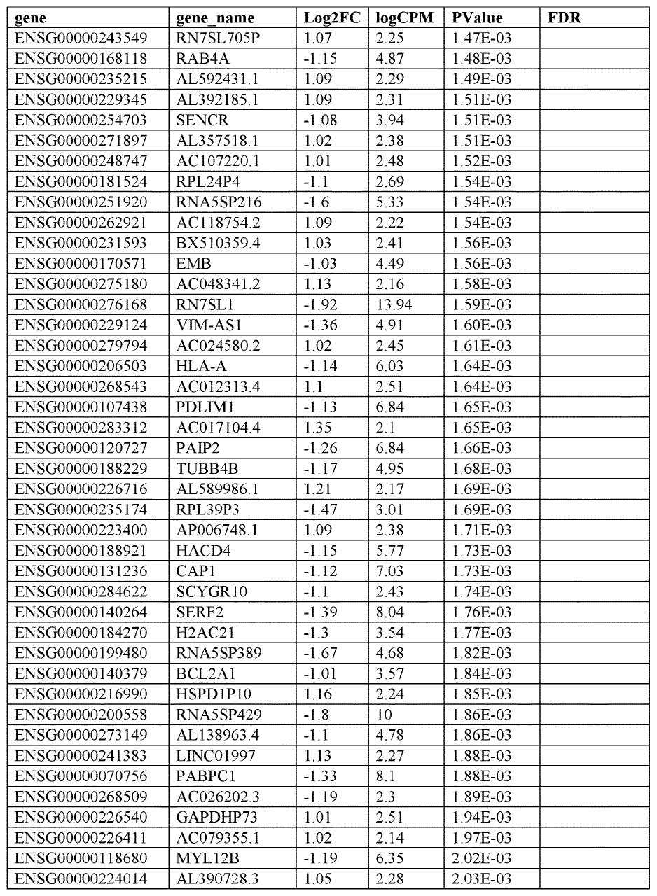

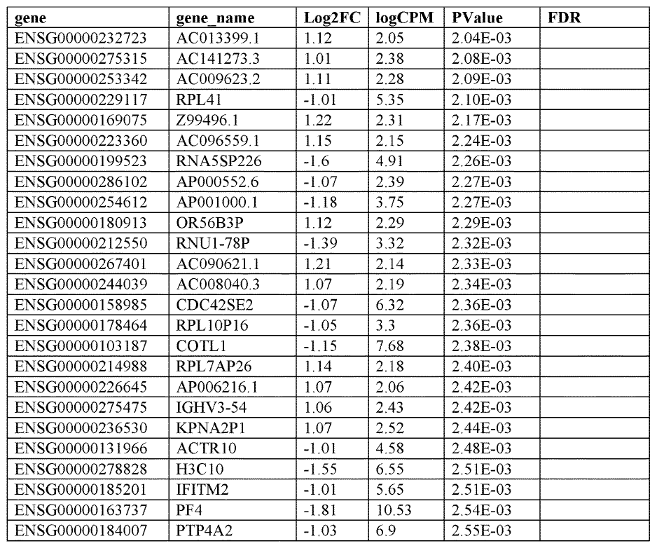

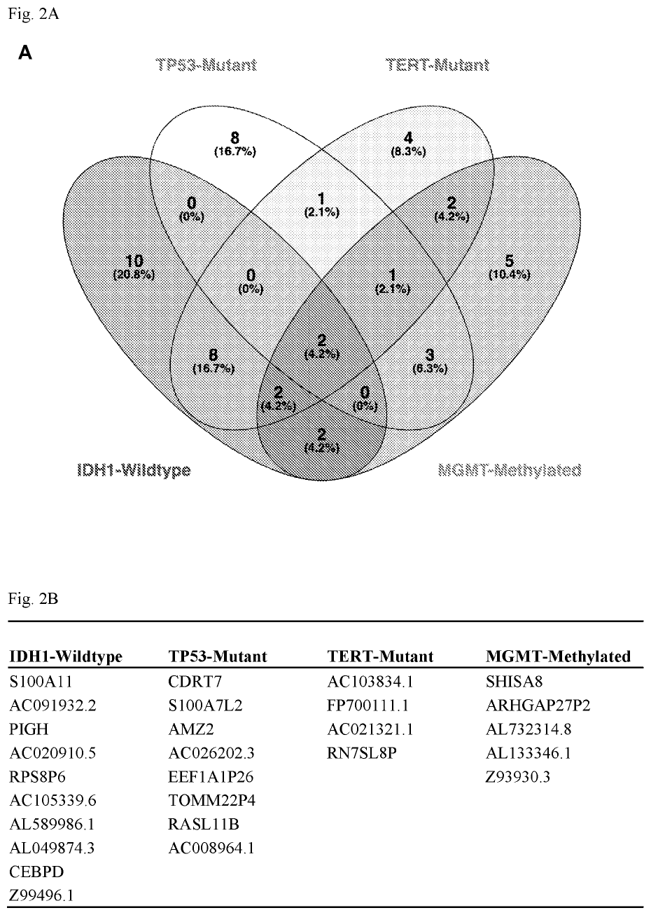

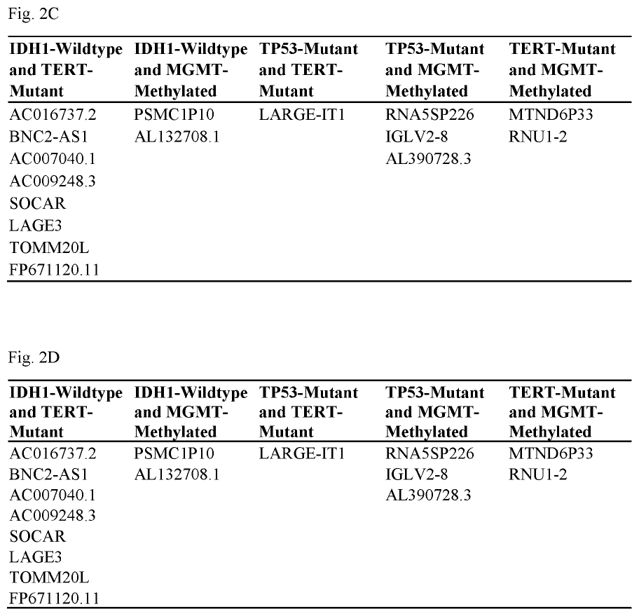

To compare the dysregulated genes found in each cancer panel, we created a Venn diagram (FIG. 2A). From this analysis, we found ten DEGs specific to IDH1-wt glioblastoma including S100A11, AC091932.2, PIGH, AC020910.5, RPS8P6, AC105339.6, AL589986.1, AL049874.3, CEBPD, and Z99496.1. In the TERT promoter mutation cancer panel we found four DEGS specific to TERT promoter mutational status including AC103834.1, FP700111.1, AC021321.1, and RN7SL8P. We found eight DEGS specific to the p53 gene mutation cancer panel including CDRT7, S100A7L2, AMZ2, AC026202.3, EEF1A1P26, TOMM22P4, RASL11B, and AC008964.1. The MGMT promoter methylation panel contained five DEGS specific to patients with MGMT methylation including SHISA8, ARHGAP27P2, AL732314.8, AL133346.1, and Z93930.3 (FIG. 2B). Several DEGS were found in multiple cancer panels. We found two genes, PSMC1P10 and AL132708.1, specific to patients with IDH1-wt, MGMT-methylated glioblastoma. Eight genes, AC016737.2, BNC2-AS1, AC007040.1, AC009248.3, SOCAR, LAGE3, TOMM20L, and FP671120.11, were specific to patients with IDH1-wt and TERT promoter mutated glioblastoma. Three genes, RNA5SP226, IGLV2-8, and AL390728.3, were specific to glioblastoma patients with p53 mutations and MGMT methylation. Two genes, MTND6P33 and RNU1-2, were specific to glioblastoma patients with TERT promoter mutations and MGMT methylation. One gene, LARGE-IT1, was specific to glioblastoma patients with mutations in the p53 gene and the TERT promoter (FIG. 2C). We found multiple DEGS that were present in three or more cancer panels. Two genes, AC017104.4 and UNC93B7, were found to be specific for patients with IDH1-wt, TERT promoter mutant, and MGMT-methylated glioblastoma. There was one gene, PNPP1, specific to glioblastoma patients with MGMT methylation, and mutations in the p53 gene and the TERT promoter. Finally, there were two DEGs, RNA5SP145 and AC108449.2, specific to patients with MGMT methylation, p53 mutation, TERT promoter mutation, and IDH1-wt glioblastoma (FIG. 2D).

### DISCUSSION

Imaging modalities and tissue biopsies have inherent limitations that render them unsuitable for use for accurate and timely diagnosis and monitoring of disease progression and treatment response. Tissue biopsies require an invasive procedure and can only offer a snapshot of tumor evolution at a single time point. Imaging modalities are insufficient to distinguish actual tumor progression from treatment artifacts that mimic progression; moreover, they require costly instrumentation and time. For brain tumors, detecting serum biomarkers once was challenging due to the blood-brain barrier (BBB), which impedes release of tumor entities into the bloodstream, even though the integrity of the BBB is compromised in cases of high-grade glioma.

Recently, circulating nucleic acids, tumor cells, and vesicles containing RNA, proteins, and lipids, such as EVs in blood and cerebrospinal fluid, have been under active investigation. Accordingly, this study focused on developing patient serum-derived EV-based glioblastoma tumor biomarker panels at the genetic level that can be used in the clinic to diagnose glioblastomas, monitor tumor burden, and assess progression and tumor response to the therapy.

Glioblastomas release EVs carrying complex biologically active molecules into the tumor microenvironment, CSF, and bloodstream, and they are thus attractive targets for biomarkers. Obtaining CSF via lumbar puncture cannot easily be justified as a routine procedure for glioblastoma diagnosis and follow-up care due to the invasiveness of the procedure and risk for brain herniation in the presence of the tumor mass effect and other detrimental complications. Obtaining serum is easier because patients with glioblastoma have increased levels of circulating EVs. Tumor-specific EVs and elevated plasma EV concentrations in glioblastoma patients were found to drop after surgery but rise again at tumor relapse, suggesting that EV dynamics might reflect disease status. Recently, tumor-specific molecules, such as EGFRvIII protein and mRNA and mutant IDH mRNA and DNA, were detected in EVs obtained from glioma cell cultures and liquid biopsies of glioblastoma patients. Although these results highlight the diagnostic potential of EVs, the informative value of mutations in a few selected genes affected by recurrent hotspot mutations, such as IDH1 or EGFR, is limited to only a subset of patients harboring these alterations. More comprehensive profiling is necessary to classify tumors with unknown genetic alterations and to monitor changes in the genetic and epigenetic tumor make-up over the course of disease treatment and progression.

Reports suggest that exosomal miRNA screening may be used as a predictive biomarker for glioblastoma patients to monitor response to chemotherapy and drug resistance, and recent studies have sought to develop a diagnostic panel to diagnose glioblastoma from serum samples. For example, Manterola et al. found increased levels of RNU6-1, miRNA-320, and miRNA-574-3p that correlated with glioblastoma diagnosis with a specificity and sensitivity of approximately 86%. Using unbiased high-throughput next-generation sequencing (NGS) and an integrative bioinformatics platform, others detected 26 differentially expressed miRNAs in glioblastoma patients compared to healthy controls. A panel of seven miRNAs, miRNA-182, miR-328-3P, miR339-5p, miR340-5p, miR-486-5p, and miR-543, able to predict glioblastoma diagnosis with 91% accuracy were selected. Using a multivariate model, four miRNAs, miRNA-182, miR-328-3P, miR340-5p, and miR-486-5p, were able to distinguish glioblastomas from healthy controls with 100% accuracy. Moreover, by using on-chip immunofluorescence to measure the concentration of GFAP and TAU proteins in EVs, it was reported that it is possible to differentiate the plasma of controls from that of glioblastoma patients, with 60% and 94% accuracy, respectively.

The present study used RNA sequencing of serum EVs isolated from a large cohort of IDH-wt glioblastoma patients and healthy controls to uncover new biological markers with prognostic and diagnostic utility. To date, the most crucial molecular marker in tumors of the CNS is IDH1/IDH2 mutations, identification of which is as an essential component for the diagnosis, characterization, and prognosis of diffuse gliomas. In general, an absence of molecular features of glioblastoma should prompt additional molecular testing (e.g., BRAF alterations, histone mutations, methylome profiling) to reach a specific diagnosis and to exclude that of other IDH-wt gliomas, such as diffuse midline glioma neuroepithelioid tumors, ganglioglioma, pleomorphic xanthoastrocytoma (PXA), and pilocytic astrocytoma. This study excluded H3K27M- or BRAF V600E-mutated IDH-wt gliomas. Diffuse midline glioma with histone H3K27M mutation, recognized as grade IV by the WHO in 2016, represents a group of diffusely infiltrating gliomas found in midline CNS structures such as the thalamus, brainstem, and spinal cord. BRAF V600E mutations are more commonly associated with other IDH-wt gliomas, such as circumscribed neuroepithelioid tumors, ganglioglioma, pleomorphic xanthoastrocytoma (PXA), and pilocytic astrocytoma. However, 54% to 93% of epithelioid glioblastomas express BRAF V600E. A gene signature is described herein that can distinguish patients with IDH-wt glioblastoma from healthy controls with high sensitivity and specificity. Indeed, the presend analysis showed 96% sensitivity and 80% specificity for 24 dysregulated genes of 569 DEGs in IDH-wt glioblastoma (FIGS. 3A-3B). Regression analysis was also performed for MGMT promoter methylation and TERT mutation status. As depicted in FIGS. 5A-5B, the MGMT promoter methylation panel showed 91% sensitivity and 73% specificity for 17 of 569 genes. As shown in FIGS. 4A-4B, a panel consisting of 20 of 569 genes detected glioblastoma TERT promoter mutation with 89% sensitivity and 100% specificity. Hence, the present panel is highly sensitive and specific for diagnosing IDH-wt glioblastoma or IDH-wt glioblastoma with molecular features according to the WHO Grade 4, 2021 classification.

### CONCLUSIONS

The present study shows that serum EV-based biomarker panels can predict/diagnose the tumor tissue status of IDH1 mutation, MGMT promoter methylation, TERT promoter mutation, and p53 mutation with high sensitivity and specificity for glioblastoma.

### Methods

### Sample Collection

The Institutional Review Board of Hacettepe University Faculty of Medicine approved the study. All participants provided written informed consent before participating in the study. The study cohort consisted of 91 glioblastoma patients (52 males, 39 females) and 31 healthy, age- and sex-matched control subjects (14 males, 17 females). Preoperative blood samples were collected in nonadditive tubes at the Department of Neurosurgery of Hacettepe University and deidentified by the Neuro-oncology Tumor Repository. Blood samples were allowed to stand at RT for 60 minutes and centrifuged at 1100×g for 15 minutes at 4° C. Serum samples were aliquoted into multiple tubes and stored at −80° C.

### Isolation and Characterization of EVs

### EV and RNA Isolation

After obtaining serum, 1 ml was exposed to RNase (6.25 μg/ml, Thermo Scientific, 12091039) for 30 minutes at RT and centrifuged at +4° C. 16000×g for 10 minutes. Supernatants were collected and loaded onto columns. TRIzol was added directly to the columns, and EVs were eluted with buffer XE. RNA from serum samples was isolated with an exoRNeasy Serum/Plasma Midi Kit (Qiagen, 77164) according to the manufacturer's instructions. The RNA concentration and quality check were performed with small RNA and Pico RNA kits. Serum (0.5, 1, and 2 ml) was used for optimization. Quantitation prior to RNA sequencing was performed with an Agilent RNA 6000 Pico Kit.

Nanoparticle Tracking Analysis (NTA) EV samples were diluted with PBS at appropriate ratios and measured with the NanoSight NS300 device (NS300, Malvern, UK). The flow-cell top plate chamber temperature was 25° C. The camera level was adjusted with video recording to minimize background noise, resulting in an image with sufficient contrast to identify particles. For each sample, five different videos of 30 seconds were prepared.

### Western Blotting

EVs were lysed in 10× RIPA buffer (Cell Signaling, Cat. No. 9806), and a bicinchoninic acid (BCA) assay was used to determine protein concentrations. Protein samples (50 μg) were separated on 10% sodium dodecyl sulfate-polyacrylamide gels and transferred to PVDF membranes. The membranes were blocked with 5% skimmed milk for 2 hours and incubated with primary CD81 (1:1000) (Novus, Cat. No. DB100-65805), TSG101 (1:1000) (Novus, Cat. No. NB200-112), and ALIX (1:200) (Santa Cruz, Cat. No. sc-53540) antibodies overnight at 4° C. After washing unbound primary antibody with 1× TBS-0.1% Tween-20, the membranes were incubated with a secondary antibody (1:100) (Novus, Cat. No. NB7511) for two hours at RT. An ECL Plus Western blotting Detection System (Cat. No. 32106, Thermo Fisher Scientific) was used for visualization.

### Enzyme-Linked Immunosorbent Assay (ELISA)

EV isolates were diluted 1:10, and a PS Capture Exosome ELISA kit (Anti-mouse IgG POD, #297-79201, Fujifilm) was used to quantify CD63 expression. The CD63 protein concentration (ng/ml) in EV isolates was calculated from a standard curve.

cDNA Synthesis

A High-Capacity cDNA Reverse Transcription Kit (Applied Biosystems) was used to synthesize cDNA from 1000 ng of total RNA. The conditions for reverse transcription were as follows: step 1, 10 min at 25° C.; step 2, 120 min at 37° C.; and step 3, 5 min at 85° C., followed by a 4° C. hold. The reaction mixture was mixed with an equal amount of RNA (10 μl).

### DNA Preparation and Bisulfite Treatment

Genomic DNA was isolated from FFPE material using the QIAamp DNA FFPE Tissue Kit (QIAGEN) according to the manufacturer's instructions.

### Pyrosequencing

The primers used in genotyping experiments are detailed in Table 6.

TABLE 6

| Primers used in genotyping | |
|---|---|
| experiments | |
| Gene | Sequence |
| TERT_forward | CCTGCCCCTTCACCTTCCAG |
| TERT_reverse | AGGACGCAGCGCTGCCTGAA-biotin |
| TERT_sequencing | ACCCCGCCCCGTCCCGACCCC |
| IDH1_forward | TGGGTGGCACGGTCTTCAG |
| IDH1_reverse | CAAAATCACATTATTGCCAACATGAC-biotin |
| IDH1_sequencing | GGGTAAAACCTATCATCATA |
| IDH2_forward | GCCCATCATCTGCAAAAACATC |
| IDH2_reverse | GGGTGAAGACCATTTTGAAAGTGC-biotin |
| IDH2_sequencing | AGCCCATCACCATTG |
| BRAF_forward | GCTTGCTCTGATAGGAAAATGAG |
| BRAF_reverse | GTAACTCAGCAGCATCTCAGG-biotin |
| BRAF_sequencing | GGTGATTTTGGTCTAGC |
| H3F3A_forward | CAATGCTGGTAGGTAAGTAAGGAGGTC |
| H3F3A_reverse | CCTGTAACGATGAGGTTTCTTCACC-biotin |
| H3F3A_sequencing | CAAAAGCCGCTCGCA |

### IDH1 and IDH2

Portions of the IDH1 and IDH2 genes were amplified, spanning codon 132 (IDH1) and codon 172 (IDH2) mutation sites, respectively. Thermal cycling consisted of 45 cycles of denaturing (95° C., 30 s), annealing (53° C., 30 s), and elongation (72° C., 30 sec) steps, preceded by an initial denaturation step (95° C., 15 min) and followed by a final elongation step (72° C., 6 min). Pyrosequencing analysis was performed with a PyroMark Q24 Qiagen system. Briefly, single-stranded DNA was prepared from 20 μl of biotinylated PCR product with streptavidin-coated Sepharose beads (GE Healthcare) and 0.4 mM sequencing primers using PyroMark Vacuum Prep Tool (Qiagen) according to the manufacturer's instructions.

### BRAF and H3F3A

Portions of the BRAF and H3F3A genes were amplified, spanning codon 600 (BRAF) and codons 27 and 34 (H3F3A) mutation sites, respectively. Thermal cycling consisted of 45 cycles of denaturing (94° C., 30 s), annealing (60° C., 45 s), and elongation (72° C., 30 s) steps, preceded by an initial denaturation step (95° C., 15 min) and followed by a final elongation step (72° C., 6 min). Pyrosequencing analysis was performed using a PyroMark Q24 Qiagen system as described above with 0.4 mM of the sequencing primers.

### TERT

A portion of the TERT promoter region was amplified, spanning nucleotide positions −228 and −250. Thermal cycling consisted of 50 cycles of denaturing (94° C., 30 s), annealing (60° C., 30 s), and elongation (72° C., 30 s) steps, preceded by an initial denaturation step (95° C., 15 min) and followed by a final elongation step (72° C., 10 min). Pyrosequencing analysis was performed using a PyroMark Q24 Qiagen system as described above with 0.4 mM of the sequencing primer.

### MGMT

An EpiTect Bisulfite Kit (QIAGEN, Cat. No. 59104) was used for bisulfite treatment of genomic DNA. To determine MGMT promoter methylation status, 50 ng of bisulfite-treated genomic DNA was analyzed using Therascreen MGMT Pyro Kit (QIAGEN, Cat. No. 971061) according to the manufacturer's instructions.

RNA Extraction from Serum EVs

One milliliter of serum sample was treated with RNase (6.25 μg/ml, Thermo Fisher Scientific, Cat. No. EN0601) for 30 minutes at RT and centrifuged at +4° C. at 16000×g for 10 minutes. Supernatants were collected and loaded onto spin columns. TRIzol was added directly to the columns, and EVs were eluted in Buffer XE (QIAGEN, Cat. No. 76214). ExoRNeasy Serum/Plasma Midi Kit (QIAGEN, Cat. No. 77144)) was used to isolate RNA from serum EVs.

### Total RNA Sequencing

Before library preparation, Agilent Small RNA (Cat. No. 5067-1548) or RNA 6000 Pico (Cat. No. 5067-1513) kits were used to assess the quality of the RNA samples. SMART-Seq Stranded Kit (Cat. No. 634444) was used to generate cDNA libraries from 2.5 ng of total RNA. To avoid loss of the limited amount of starting material, ribosomal RNA (rRNA) depletion was performed before the final PCR amplification step. A High Sensitivity DNA Kit (Agilent Technologies, Santa Clara, USA) was used to verify the size distribution of the sequencing-ready libraries. The cDNA libraries were quantified with the Qubit High Sensitivity DNA kit (Invitrogen, Thermo Fisher Scientific, USA). Equal amounts of indexed 300-400 bp libraries were pooled and paired-end sequenced with HiSeq 2500 and NovaSeq 6000 sequencers (Illumina).

### Analysis of RNA-Seq Data

RNA sequence data analysis, ROC curve preparation, and LASSO graphs were generated.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A panel of probes that hybridize to nucleic acid, the panel comprising
(a) at least 2 probes specific for IDH1-wt or IDH1 R132H selected from the group consisting of AC108449.2, RNA5SP145, FP671120.11, AC016737.2, AC017104.4, RPS8P6, AC105339.6, BNC2-AS1, AC007040.1, AC091932.2, AL589986.1, PSMC1P10, AC009248.3, SOCAR, S100A11, PIGH, AL132708.1, LAGE3, AC020910.5, TOMM20L, AL049874.3, UNC93B7, CEBPD, and Z99496.1;
(b) at least 2 probes specific for a p53 mutation selected from the group consisting of RNA5SP226, AL390728.3, CDRT7, RNA5SP145, AC108449.2, PNPP1, S100A7L2, AMZ2, AC026202.3, EEF1A1P26, LARGE-IT1, TOMM22P4, RASL11B, IGLV2-8, and AC008964.1;
(c) at least 2 probes specific for MGMT promoter methylation selected from the group consisting of RNA5SP226, IGLV2-8, MTND6P33, AC108449.2, SHISA8, ARHGAP27P2, AL390728.3, PSMC1P10, AC017104.4, UNC93B7, AL732314.8, AL133346.1, PNPP1, AL132708.1, RNU1-2, RNA5SP145, and Z93930.3: or
(d) at least 2 probes specific for TERT promoter mutation selected from the group consisting of BNC2-AS1, AC017104.4, AC009248.3, AC108449, AC007040.1, RNA5SP145, LAGE3, AC103834.1, UNC93B7, AC016737.2, PNPP1, MTND6P33, FP671120.11, FP700111.1, TOMM20L, AC021321.1, SOCAR, RN7SL8P, RNU1-2, and LARGE-IT1.

2-5. (canceled)

6. The panel of probes of claim 1, wherein the panel comprises at least 3 probes.

7. The panel of probes of claim 1, wherein each probe comprises a unique label.

8. A kit comprising a collection of probes, wherein the collection comprises a panel of probes of claim 1, and instructions for use in analyzing a biological sample.

9. The kit of claim 8, wherein the biological sample is a liquid biopsy.

10. The kit of claim 9, wherein the liquid biopsy is blood or a blood product.

11. A method of detecting the presence of biomarkers associated with an increased risk of glioblastoma in a human subject, comprising:
(i) contacting DNA from a biological sample containing cells from the subject with the panel of probes of claim 1 to form hybridized target sequences;
(ii) detecting the hybridized target sequences;
(iii) determining the number of hybridized target sequences detected; and
(iv) indicating that the human subject has an increased risk of glioblastoma if more than 1 biomarkers are present.

12. The method of claim 11, wherein the biological sample is a liquid biopsy.

13. The method of claim 12, wherein the liquid biopsy is blood or a blood product.

14. The method of claim 1, wherein the biological sample is subdivided into individual subsamples, and a different single probe is applied to each subsample.

15. A method of treating a human subject for glioblastoma, comprising:
(i) contacting DNA from a biological sample containing cells from the subject with the panel of probes of claim 1 to form hybridized target sequences;
(ii) detecting the hybridized target sequences;
(iii) determining the number of hybridized target sequences detected;
(iv) indicating that the human subject has an increased risk of glioblastoma if more than 1 biomarkers are present; and
(v) administering an appropriate treatment to the patient.

16. (canceled)

17. The method of claim 15, wherein the panel comprises at least 3 probes.

18. The method of claim 1, wherein the treatment comprises surgery followed by IR and chemotherapy.

19. A method of detecting glioblastoma in a patient comprising:
(a) obtaining a sample from the patient;
(b) isolating extracellular vesicles from the sample; and
(c) detecting expression of at least one cancer marker in the isolated extracellular vesicles.

20. The method of claim 19, wherein step (b) extracellular vesicles are isolated by ultracentrifugation.

21. The method of claim 19, wherein the extracellular vesicles are exosomes.

22. The method of claim 21, wherein the extracellular vesicles are about 30 to about 150 nm in size.

23. The method of claim 19, wherein the extracellular vesicles express at least one of the exosomal markers IDH1-wt or IDH1 R132H, a p53 mutation, MGMT methylation, or TERT promoter mutation.

24. The method of claim 23, wherein the exosomal markers IDH1-wt or IDH1 R132H, a p53 mutation, MGMT methylation, or TERT promoter mutation.

25. The method of claim 19, wherein in step (c), expression of the at least one cancer marker is detected using a micro flow cytometer.

26. The method of claim 19, wherein step (c) comprises determining a differential expression profile for at least one cancer marker in the sample as compared to a control.

27. The method of claim 19, wherein the markers on individual extracellular vesicles within the isolated extracellular vesicles are detected.

28. The method of claim 19, wherein the sample is a body fluid sample or a tissue sample.
